(19) United States
(12) Patent Application Publication
(10) Pub. No.: US 2026/0229324 A1
(43) Pub. Date: Aug. 6, 2026

(54) Using Artificial Intelligence/Machine Learning for Remote Monitoring and Assisting of Individuals Under Care, and Systems and Methods Therefor

(71) Applicant: Therap Services, LLC, Torrington, CT (US)

(72) Inventors: James Michael Kelly, Morris, CT (US); David Lawrence Turock, Fort Lauderdale, FL (US); Justin Mark Brockie, Wolcott, CT (US); Md Asif Ali, Rocky Hill, CT (US); Meheraj Hossain, Dhaka (BD); Raheeb Hassan, Dhaka (BD); Md Nazrul Islam, Dhaka (BD); Rafid Ameer Mahmud, Dhaka (BD); Pranjal Kumar Nandi, Dhaka (BD); Richard Allen Robbins, Lenox, MA (US); William Edward Sepesi, San Francisco, CA (US); Md Rayed Bin Wahed, Dhaka (BD); James Joseph Brockman, Millstone Township, NJ (US)

(73) Assignee: Therap Services, LLC, Torrington, CT (US)

(21) Appl. No.: 19/222,011

(22) Filed: May 29, 2025

Related U.S. Application Data
(60) Provisional application No. 63/751,943, filed on Jan. 31, 2025.

(51) Int. Cl. G16H 10/60; G06F 40/279; G06V 10/74; G06V 20/40; G06V 40/10

(52) U.S. Cl. CPC G16H 10/60; G06F 40/279; G06V 10/74; G06V 20/41; G06V 20/44; G06V 40/10

(57) ABSTRACT

Systems and methods for remote monitoring and assisting of individuals under care by a caregiver, using artificial intelligence/machine learning, include a HIPAA-compliant computer system having an event database of stored textual descriptions of events, an event-to-action database of action plans corresponding to the events, and a location having sensors for collecting data regarding the individual. The computer system creates textual descriptions of the sensor data and combines them into a unified textual description. This description is compared to the stored descriptions and if they match or are similar, an action corresponding to the event is identified; otherwise, input identifying an action corresponding event is accepted and stored in the event-to-action database. An action, such as sending an alert to the caregiver, is performed. The caregiver receives the alert if the caregiver's caseload permits access to the individual's information.

### CROSS-REFERENCE TO RELATED APPLICATIONS

This application claims, and is entitled to claim, a right of priority to U.S. Provisional Patent Application Ser. No. 63/751,943 filed Jan. 31, 2025, and is entitled to the benefit of the filing date thereof.

This application incorporates the entirety of the following U.S. Patents and Patent Applications:
U.S. Pat. No. 12,217,316, issued Feb. 4, 2025 (“the '316 Patent”) filed as U.S. patent application Ser. No. 17/827,521 on May 27, 2022 and published as U.S. Patent Publication No. US 2022/0301074 A1 on Sep. 22, 2022 (“the '074 Publication”);
U.S. Pat. No. 11,915,806, filed as U.S. patent application Ser. No. 17/941,329 on Sep. 9, 2022 (“the '806 Patent”);
U.S. Pat. No. 11,475,983 filed as U.S. patent application Ser. No. 16/695,591, on Nov. 25, 2019 (“the '983 Patent”);
U.S. Pat. No. 8,281,370 filed as U.S. patent application Ser. No. 11/604,577, on Nov. 27, 2006 (“the '370 Patent”).
U.S. Provisional Patent Application Ser. No. 63/751,354 filed Jan. 30, 2025 (“the '354 Application”).

The '316 Patent is a continuation-in-part of U.S. Pat. No. 11,449,954 filed as U.S. patent application Ser. No. 16/750,388 on Jan. 23, 2020, which is a continuation-in-part of U.S. Pat. No. 10,586,290 filed as U.S. patent application Ser. No. 15/197,120 on Jun. 29, 2016, which is a continuation-in-part of U.S. patent application Ser. No. 13/675,440 (“the '440 Application”) filed Nov. 13, 2012. The '316 Patent is also a continuation-in-part of U.S. patent Ser. No. 11/410,759 filed as U.S. patent application Ser. No. 16/811,429 on Mar. 3, 2020, which is a continuation of U.S. Pat. No. 10,622,103 filed as U.S. patent application Ser. No. 15/636,826 on Jun. 6, 2017.

The '806 patent is a continuation of the '983 Patent, which claims priority to the '440 Application, which is a continuation-in-part of U.S. Pat. Nos. 8,615,790 and 8,813,054, both of which are divisions of the '370 patent.

All description, drawings and teachings set forth in the '316, '806, '983, and '370 patents and the '354 Application are expressly incorporated by reference herein.

### BACKGROUND OF THE INVENTION

Technology at its best can help all sorts of people. Advances in technology at a societal level have helped cure diseases, allowed families to stay in touch at long distance, automated workforce processes to often eliminate jobs which caused injuries to workers, cleaned up rivers, and improved medicines and the food supply. The world increasingly relies on technology for communications, information, and automation of tasks. There are now self-driving cars, home automation where lights can turn on automatically and music can be played based on personalized worklists, facial recognition for security and so much more. While there are people who have concerns about the ethical use of technology, when advances in technology work there are often real benefits for people. Faster checkout lines, remote banking, hopefully more likelihood you will get your actual luggage when arriving at an airport and so much more.

However, when technology does not work properly there can be all sorts of issues. As technology including software, hardware, artificial intelligence, machine learning has created often larger and larger companies with dominant market share, problems with downtime and/or technology not working has shown the potential to affect large amounts of people who might otherwise be unaware that they are even affected by a specific product until there is a problem with it. In 1965 there was an electricity blackout in the United States and Canada which may have left over 30 million people without electricity and according to Wikipedia the “cause of the failure was the setting of a protective relay on one of the transmission lines.” Essentially a “mis-programmed” setting caused a massive blackout. Recently there was a massive disruption of air travel according to AP News “At the heart of the massive disruption is CrowdStrike, a cybersecurity firm that provides software to scores of companies worldwide. The company says the problem occurred when it deployed a faulty update to computers running Microsoft Windows, noting that the issue behind the outage was not a security incident or cyberattack.” According to the New York Times “Airlines grounded flights. Operators of 911 lines could not respond to emergencies. Hospitals canceled surgeries. Retailers closed for the day. And the actions all traced back to a batch of bad computer code.”

Most of these articles and technologies focus on large scale outages and how people or systems had the capabilities to respond and what actions they took. However, there are groups of individuals in society who may not have the intellectual capabilities to recognize what happened and take action to mitigate damage or potential problems. There are significant populations of people who are elderly, with intellectual or developmental disabilities, with dementia or other cognitive disabilities and others receiving home and community-based services. These individuals historically had relied on other human beings to provide support. These individuals could have received support from direct care givers, direct support professionals, nurses, home care aides, family members, and other individuals providing in-person direct support to these individuals. These people providing direct support could have been provided funding from governments, non-profits, insurance companies, family members, even the individuals themselves, or provided their service at no cost. But historically the service and assistance was provided directly, generally without the assistance of sophisticated technology. There might have been wheelchair lifts or similar devices, but there still would have been people present to operate them.

What is changing is that as there is an increased movement to community based services, and a reliance on using technology to enhance the independence of people with intellectual disabilities to live independently while still receiving supports from human staff and family who may or may not be awake, in person, and watching what is happening in person in real time, there is a risk that if there are failures in technology on either a small scale (for example just the device in someone's room) to a large scale (electricity blackouts or cellphone or internet outages) many products being created today by consumer product companies or even B2B or other companies are not being designed to think about multiple points of failure for some of the most vulnerable people.

The May 10, 2024 Federal Register final rule from the Federal Department of Health and Human Services on page 40577 lists some of the consumer rights for people supported, these included “control over the individual's own physical environment, living and sleeping space,” “control over personal resources,” and “right to privacy, dignity and respect,” among other objectives. Developments in technology are helping and will increasingly help individuals achieve these objectives. The non-profits, governments and other organizations providing supports are utilizing these technologies to comply with regulations, manage workforce challenges and meet individuals' goals and objectives.

For example, a sleep mat which monitors sleep and notifies a caregiver in the morning how someone slept is a somewhat simple example of information which can be remotely obtained. In this example if the sleep mat does not convey the information to the staff in the morning, but the staff if still present, the staff can use in person observations to overcome the lack of information from technology.

But what happens, for example if the sleep mat is designed to awaken a sleeping staff member if someone gets out of bed in the middle of the night? This is a different issue as there could be a real-time flight risk or known safety risk with a particular individual getting out of bed. Perhaps before technology the staff would be sitting in the hallway all night, but with technology they could be alerted and awakened when sleeping. What happens if the technology stops working? Or when there is a blackout? Or it was supposed to rely on information being sent back from an off-site location which was not able to communicate.

There is a related issue of what happens if the technology is working and the staff member dies in the middle of the night. To some extent the human staff member or support dying can be thought of as a system failure which also would preferably be addressed.

Aspects of the present invention provide a system to essentially deal with unlikely events which could occur and have significant negative medical or other outcomes for individuals with various cognitive or other conditions.

### SUMMARY OF THE INVENTION

Aspects of the present invention provide a system for managing actions and documentation and other information from individuals under care among multiple security domains and caseloads using sensors and other devices. These individuals can include those with cognitive disabilities, prisoners, children in school, infants aged three to three, the elderly, and others. Embodiments of this invention may have applications for analysis of actions in a location by devices, sensors and machines either in addition to or in conjunction with human staff, guardians or others; may have automated actions guided by information and analysis, may have the ability to confirm information provided by sensors or computers, may have the ability to summon human involvement to a situation in compliance with PHI across and within organizations in the human service field in compliance with federal and state regulations such as HIPAA and the Cares Act, and as further described in the '983 Patent at 2:64-3:5, and the '074 Publication at Paragraphs 0010 and 0015 (collectively “HIPAA-type regulations”), as well as security procedures and objectives of furthering person-centered-thinking.

Embodiments of this system ensure that as individuals and agencies rely on various sensors, computers and automated systems for collection of data as well as actions to be taken, that procedures and processes can be in place to automatically determine that the overall flow of information is intact and working and if not to automatically either direct another sensor or device to take over the problem or else to awaken an appropriate person to help maintain the safety and care of the individual being supported.

In particular there has been a proliferation of consumer devices which can capture information. These devices can include wearables such as watches and rings, non-intrusive devices such as sleep mats and scales, and remote options such as cameras and other sensors. The 2024 Consumer Electronics Show website states that they had over 4300 registered exhibitors and many exhibitors had multiple products or devices. Many, if not, most of these products are designed for individuals, families, or small groups of users. Depending on the type of information shared, in many cases the data or information could be some sort of family or friend group. For example, if people are sharing their steps or workout routine they might feel comfortable sharing this with acquaintances, or even people they do not know, who share similar interests. For medical information such as blood glucose, blood pressure or heart rates, people may keep the information only to themselves or a small group of family, medical professionals, and/or close friends. This information can be challenging for individuals to manage, which has led to many websites and services providing individuals and families with tools to manage the data within a group.

In addition to the issue of managing the data, there is an issue what happens when a system goes down or a process stops working. For some people, and not only those who could be defined as neuro-normative, there could be an ability to either handle either hardware or software systems when they stop working or at least adjust their behavior to mitigate any immediate danger. For example, if someone's internet, electricity, and/or phone connectivity stops working they might make an assessment of how they are feeling and what adjustments need to be made because Wi-Fi or internet for example are not available. A person might not need to know how to fix access to devices but could be able to make independent decisions to increase their safety. There are certain devices such as a Dexcom Blood Glucose monitor which could have built in medication delivery systems into their process. These devices might be designed to provide an analysis and action within a given device. Those devices may have independent or built in methods of dealing with battery outages or other forms of the device not working. The embodiments of this invention relate to a comprehensive system with, potentially, information from multiple sensors being analyzed in a person-centered manner where data, information and actions taken could cross multiple locations and devices.

Much of the information that individuals and/or caregivers are documenting falls into some sort of useful-to-have, but not life-threatening, if it is not available in the short term. For example, people with food allergies can typically manage their own diets if a diet tracker or automated nutrition app became unavailable. They might be able to recognize that the information is unavailable and make a decision on their own to exercise personal human judgement. For example, they could read the labels on food or eat only foods they are previously aware are safe to eat. In another example, when a carbon-dioxide monitor or smoke alarm stops working, many devices now are set up to make a localized noise until someone within hearing distance perhaps changes the battery. So, in effect for individuals or small units who are able to analyze and take action on certain information, many consumer products are designed both for a small, localized group to be able to take action as well as building in a level product downtime or non-performance.

However, for individuals with intellectual, developmental, or other cognitive disabilities who are receiving various types of home and community-based support, there is a need to think about the flow of information regarding an individual receiving supports to their staff, circle of support or management of their support. Individuals could live in independent living situations but receive remote monitoring and support, they could live in small community-based homes with 2 to 5 or more individuals or even for certain populations such as portions of the elderly there could be 100 or more individuals in a managed location. These multiple individuals could have multiple staff on shift at any given time. And there are multiple shifts throughout the day. In addition to paid staff there could be family members, guardians, volunteers, friends, or others who might help provide supports and need access to information.

For people with physical disabilities and/or in addition to cognitive disabilities there could be a situation where they do not have the physical capacity to take action. There could for example be in a wheelchair and unable to independently change the battery on a ceiling smoke detector.

There is a need for the right people or even the right automated device or system to have access to information. As society is moving to more sensor and devices-based tracking of information, there could be a problem with the overall system of information flows when a series of multiple malfunctions and problems simultaneously occur. For example, the planned support monitoring for an individual sleeping at night who was a fall or flight risk who had an electronic device (whether radar, thermo, IR or other) was to have that device send information out of the room to a cloud based analytics or computer system (in particular one outside of the residence location that the person is living in) using artificial intelligence, machine learning, care plans and the history of person centered data about that individual, and based on that information could either send an alert to an sleeping overnight staff member who might be on-call to wake when certain problem scenarios arise. In this scenario the staff member would receive a message possibly through an alarm or a vibration and wake up and deal with the situation. In addition, the system could analyze the situation and turn on lights, lock certain doors, activate noises, or alert police or other external supports. All of this could be done according to a person-centered plan for outcomes and goals and be respectful and aware of human rights committee guidelines for that specific individual.

In the scenario just described the information could be generated by say a device connected to electricity, transmitted within the home by Wi-Fi, then transmitted to an outside location for computing by internet, processed at the off-site location requiring electricity and internet, and then sent back to the staff on duty using either cellphone or internet. In addition, many agencies with overnight staff might encourage their staff not to bring their cell phones to work to avoid distractions so they are relying even more on the internet. If this string of electricity and internet were to stop working, the staff member sleeping down the hall would not have processed information to know when to wake up to assist an individual requiring care.

Aspects of this invention are designed to assist in situations where flows of information are uncertain or stopped and allow situations with many caregivers to many individuals, and have methods of knowing if required information is received by appropriate people or systems, and if not, to essentially automatically activate backup processes or procedures.

Another potentially important part of this process is the need to track and document what has occurred. There are significant numbers of individuals that are receiving federal, state, local, funded, insurance, or other funding. The agencies and/or caregivers not only need to provide a service, but document what services were provided and why they were provided. This system can include the ability to document decisions, notifications and show proof or verification that information was not only conveyed but also received by appropriate people.

An exception to products which have built-in downtime or non-performance might be a continuous blood glucose device such as Dexcom which is also connected to an automated insulin delivery system such as a connected pump. At present this product appears to need a prescription and is treated as a medication delivery tool.

Embodiments of this system provide for appropriate documentation under HIPAA-type regulations. When working with systems with federal and state funding there are often heightened requirements for proof of documentation. This can include regulations and requirements such as Electronic Visit Verification, proof of service delivery, proof of staffing capabilities and requirements, compliance with state and federal regulations, compliance with insurance industry or funding requirements and other needs for documentation. This system can provide documentation in various locations including at point of service as well as off site.

While normally there can be the ability to have information about events including falls, in/out of bed, incontinence and other incidents or events transferred in real time to an off-site location. And once data is in that other off-site location it cannot be deleted without a strict set of rules. This could mean that if an onsite device captured abuse and neglect or physical or emotional violence towards an individual the person instigating that abuse would not be able to adjust the data stores in the system.

In instances where the internet and ability to transfer the data off-site were down due to internet, Wi-Fi or other means of transferring data and information off-site, there could still be the ability to securely store the data on-site through devices, storage arrays, computers and other methods of storage which a staff or visitor would not be able to tamper with or adjust; the information could then be transmitted to the secure store when power and internet connectivity were restored.

There is also the possibility that a person could create an incident and recognize that they would eventually be caught even with backup data through a house or other location.

Embodiments of this system provide a device which can be protected against fire and also have the ability to send out a notification to be found in the instance where the equipment is targeted for intentional damage.

Embodiments of this system are built upon the concept of caseloads and super roles previously described in the '370 Patent at Col. 5, Line 21-Col. 7, Line 32; Col. 12, Line 1-Col. 13, Line 8; Col. 22, Lines 30-59, Col. 24, Lines 40-57 (caseloads) and in the '370 Patent at Col. 13, Line 4-Col. 15, Line 10; Col. 22, lines 30-59 (roles and super roles).

Embodiments of this system provide for physical devices with the ability to automatically wake an individual if certain conditions such as internet access, electrical service, phone service, working sensors or other conditions are not active. There are situations where one of several conditions might not be a problem but combined they could be and the device would have instructions on what to do in location if this occurred. An example of this is if due to snow or windstorm in a rural community where internet connections, power, and cellphone were all simultaneously not working. In that situation, an individual with cognitive disabilities who was being monitored by sensors in their home bedroom might send a notice to a staff member sleeping in a separate room to wake up and do automatic monitoring.

This individual's information would be securely obtained and stored, reducing the risk of loss of information (understanding that completely eliminating any risk to a probability of zero is next to impossible) as well as tampering, deleting, and having foreign or unwanted actors manipulating the data.

The systems and methods disclosed as embodiments of the invention improve the ability of caregivers to respond to an event, occurring in a location where an individual under care is present, in a HIPAA-compliant way.

Systems and devices that perform the functions at least of: (1) providing an event database of stored textual descriptions of events; (2) providing an event-to-action database of action plans corresponding to the events; (3) providing a location having sensors for collecting data regarding an individual; (4) collecting data from the sensors; (5) creating textual descriptions of the data collected by the sensors; (6) combining the textual descriptions into a unified textual description; (7) comparing the unified textual description to the stored textual descriptions; (8) identifying whether the unified text description matches a stored textual description; identifying a matching event, and identifying an action corresponding to the matching event; (9) identifying whether the unified textual description is similar to a stored textual description, identifying a similar event, and identifying an action corresponding to the similar event; (10) identifying whether the unified text description does not match or is similar to a stored textual description, accepting a training input identifying a non-matched event, accepting a training input identifying an action corresponding to the non-matched event; and storing the non-matching event and the action corresponding to the non-matching event in the event-to action database; and (11) performing the action, were and are neither routine, well-understood, nor conventional in the field of HIPAA-compliant patient monitoring.

Systems and devices that perform the above functions, as well as the functions at least of: (1) as part of performing the action, sending an alert to a device; (2) as part of identifying a matching event, identifying the individual; and (3) as part of identifying a similar event, identifying the individual, were and are neither routine, well-understood, nor conventional in the field of HIPAA-compliant patient monitoring.

### BRIEF DESCRIPTION OF DRAWINGS

FIG. 1 depicts an overview of the embodiments of the present invention showing the flow of information and supports in a monitored location.

FIG. 2 depicts an overview of a monitored location.

FIG. 3 depicts a residential facility.

FIG. 3A depicts a sample home floor plan.

FIG. 4 depicts a monitoring system.

FIG. 5A depicts the images, videos, sounds, and other data processed into texts by the Inference Action Module.

FIG. 5B depicts the Inference Action Module going to the Master Event Resolution Module and the Event-to-Action Database.

FIG. 5C depicts a workflow of the Master Event Resolution Module.

FIG. 5D depicts performed stored actions creating different alerts.

FIG. 5E depicts execution of similar action or stored training in the Event-to-Action Database.

FIG. 5F depicts using word counts similarity and keywords to identify an event.

FIG. 5G depicts a workflow for identifying an event by the Inference Action Module.

FIG. 6 depicts problems which can occur, and situations possibly requiring action.

FIG. 7 depicts community locations which might utilize aspects of the invention.

FIG. 8 depicts a circle of support.

FIG. 9 depicts cognitive conditions.

FIG. 10 depicts a school which might utilize aspects of the invention.

FIG. 11 depicts a job which might utilize aspects of the invention.

FIG. 12 depicts a leisure activity which might utilize aspects of the invention.

FIG. 13 depicts sensors, and other devices, which may be used in embodiments of the invention.

FIG. 14 depicts a bedroom as implemented in embodiments of the invention.

FIG. 14A depicts an individual asleep in a bedroom as implemented in embodiments of the invention.

FIG. 14B depicts an individual awakened in a bedroom as implemented in embodiments of the invention.

FIG. 14C depicts an individual slipping and falling out of a bed in a bedroom as implemented in embodiments of the invention.

FIG. 14D depicts an individual who has fallen to the floor of a bedroom as implemented in embodiments of the invention.

FIG. 14E depicts staff receiving an alert regarding an individual who has fallen to the floor of a bedroom as implemented in embodiments of the invention.

FIG. 14F depicts staff responding to an alert regarding an individual who has fallen to the floor of a bedroom as implemented in embodiments of the invention.

FIG. 15 depicts a bathroom as implemented in embodiments of the invention.

FIG. 15A depicts an individual brushing teeth in a bathroom as implemented in embodiments of the invention.

FIG. 15B depicts an individual spilling water in a bathroom as implemented in embodiments of the invention.

FIG. 15C depicts an individual slipping on spilled water in a bathroom as implemented in embodiments of the invention.

FIG. 15D depicts an individual who has fallen to the floor of a bathroom as implemented in embodiments of the invention.

FIG. 15E depicts staff receiving an alert regarding an individual who has fallen to the floor of a bathroom as implemented in embodiments of the invention.

FIG. 15F depicts staff responding to an alert regarding an individual who has fallen to the floor of a bathroom as implemented in embodiments of the invention.

FIG. 16 depicts communal residential areas.

FIG. 16A shows individuals playing a game in a communal residential area as implemented in embodiments of the invention.

FIG. 16B shows an individual getting angry while playing a game with another individual in a communal residential area as implemented in embodiments of the invention.

FIG. 16C depicts staff receiving an alert regarding the hurt individual in a communal residential area as implemented in embodiments of the invention.

FIG. 16D depicts staff responding to an alert regarding the hurt individual in a communal residential area as implemented in embodiments of the invention.

FIG. 17 depicts a bathroom as implemented in embodiments of the invention.

FIG. 17A depicts an individual using a knife in a kitchen as implemented in embodiments of the invention.

FIG. 17B depicts an individual cut by a knife in a kitchen as implemented in embodiments of the invention.

FIG. 17C depicts staff receiving an alert regarding an individual cut by a knife in a kitchen as implemented in embodiments of the invention.

FIG. 17D depicts staff responding to an alert regarding an individual cut by a knife in a kitchen as implemented in embodiments of the invention.

FIG. 18 depicts a living room as implemented in embodiments of the invention.

FIG. 19 depicts a garage as implemented in embodiments of the invention.

FIG. 20 depicts automation that may be implemented in embodiments of the invention.

FIG. 21 depicts alerting and notification that may be implemented in embodiments of the invention.

FIG. 22A shows an electronic form format for creating a General Event Report.

FIG. 22B shows General Event Report dropdown fields for receiving information generated as part of this invention.

FIG. 23A shows an electronic form format for creating a T-Log.

FIG. 23B shows T-Log dropdown fields for receiving information generated as part of this invention.

### DETAILED DESCRIPTION OF THE INVENTION

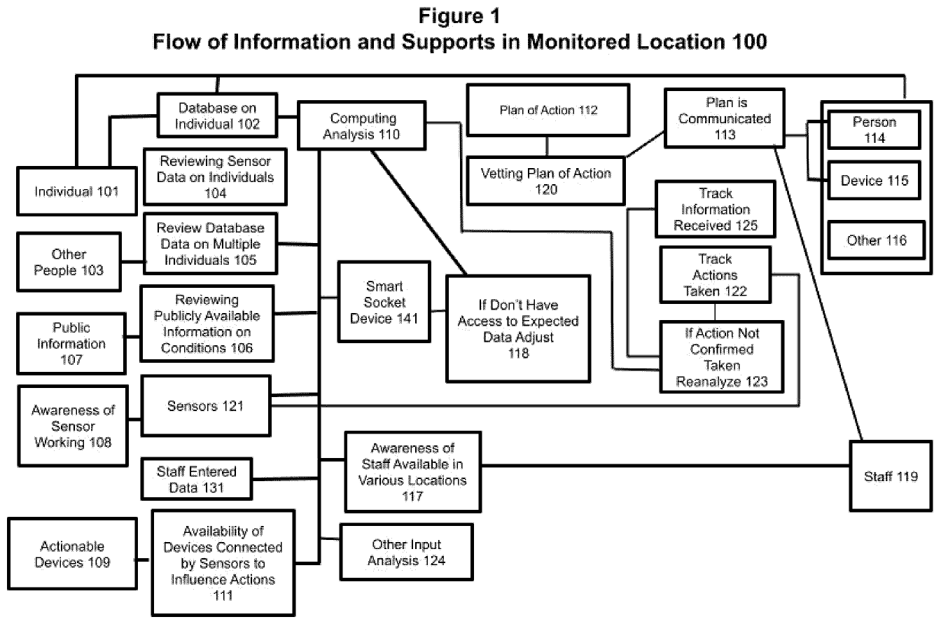

FIG. 1 depicts an overview of the embodiments of the present invention showing the flow of information and supports in a monitored location.

FIG. 1 illustrates an overview of the embodiments of the present invention system, showing the Flow of Information and Supports in Monitored Location 100. Embodiments of the present invention require a number of steps in order to fully grasp, obtain and understand the information, process, and analysis.

As shown in FIG. 1, Computing Analysis 110 receives information and data is obtained from a location using various Sensors 121, Devices 109, and databases on the individual 102, other individuals 105, and publicly available information 106. This Computing Analysis 110 will generate a Plan of Action 112. The Plan of Action 112 could include simply recording the current state of events and taking no interventional action; conversely, an action or actions can be suggested. If all systems of generating data are working properly, the system could determine if there are any notifications of staff, adjustment of devices or other actions to take to either improve quality of support, improve outcomes, or reduce risk of negative outcomes.

For example, if the Sensor 121 information is showing that at a bedroom which is the venue or monitored location (as discussed in more detail below), an Individual 101 has left the bed and is walking around the room for more than say 30 seconds, a plan of action could be to wake up staff in another room and check on the Individual or could be to play soothing music on a music system in the room which is known to cause the individual to return to Bed. These could be Individual specific Plans of Actions 112. These could be derived in whole or in part from individual care plans.

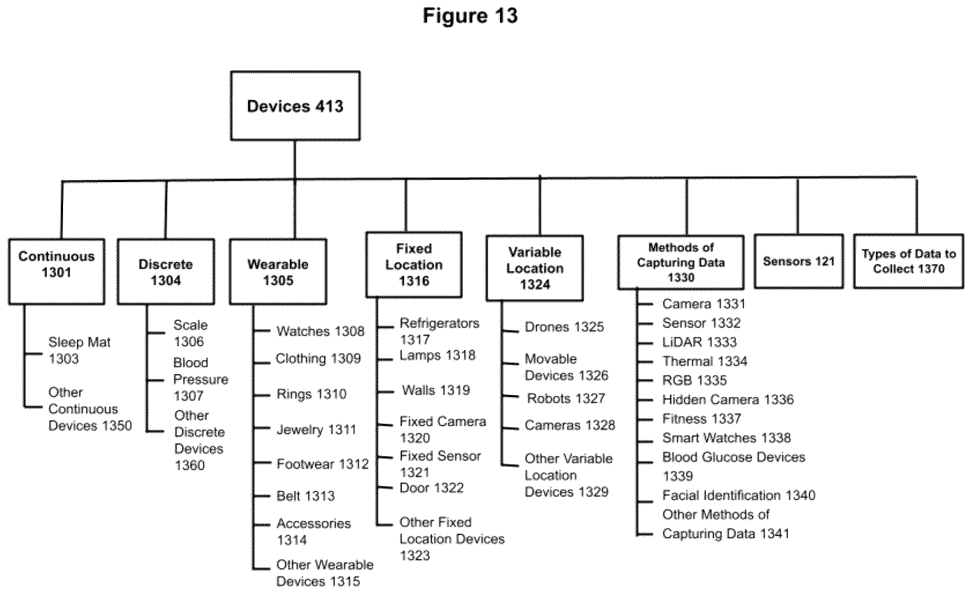

Computing Analysis 110 can include a variety of pieces of information or data. These can be from Sensors 121 and other devices (see FIG. 13), from Public Information 125, Database on Individual 102, Staff Entered Data 131, Information on Other People 103, Reviewing Sensor Data on Individuals 104, Review Database Data on Multiple Individuals 105, Reviewing Publicly Available Information on Conditions 106, Computing Analysis 110, Availability of devices connected by sensors to influence actions 111, Plan of Action 112, Awareness of staff available in various locations 117, If don't have Access to Expected Data Adjust 118, If Action not Confirmed Taken Reanalyze 123, Other input analysis 124, and Smart Socket Device 141.

The system could be performing one or more Computing Analysis 110 in one or more computing venues or locations. As discussed in more detail below, these locations could include inside the room that Individual 101 is in. This venue could be a car, a job location, a bedroom, bathroom, kitchen, a community facility, a recreation location, or any other venue or location. If the Computing Analysis 110 is performed by a computer or other device inside the same room an Individual 101 is in, this could be considered edge computing or fog computing.

A preferred machine learning system may translate pixels, sounds, and other inputs into a language-based description of the activity represented by that input. We are using words such as edge computing, fog computing, cloud computers and other words in order to separate and define locations where analysis and computing is done relative to an Individual 101 and the location of the individual. Edge computing may refer to a configuration where that translation is performed by processing machinery (i.e., hardware and software) that are embedded in the sensor(s) that acquired the information. For example, when a device containing a camera also contains computational machinery that executes machine learning instructions to translate the images that the camera has captured. Fog computing may describe a configuration where that computation is performed, either in whole or in part, by a computational facility that resides elsewhere in a physically proximate location, e.g., when a camera is in one room of a house, and the processing of the camera's images are performed elsewhere in the house. As an example, cloud processing is when input data are sent via the Internet to a cloud computing facility and the processing of that data is performed by computational machinery physically located at the cloud service providers' facility.

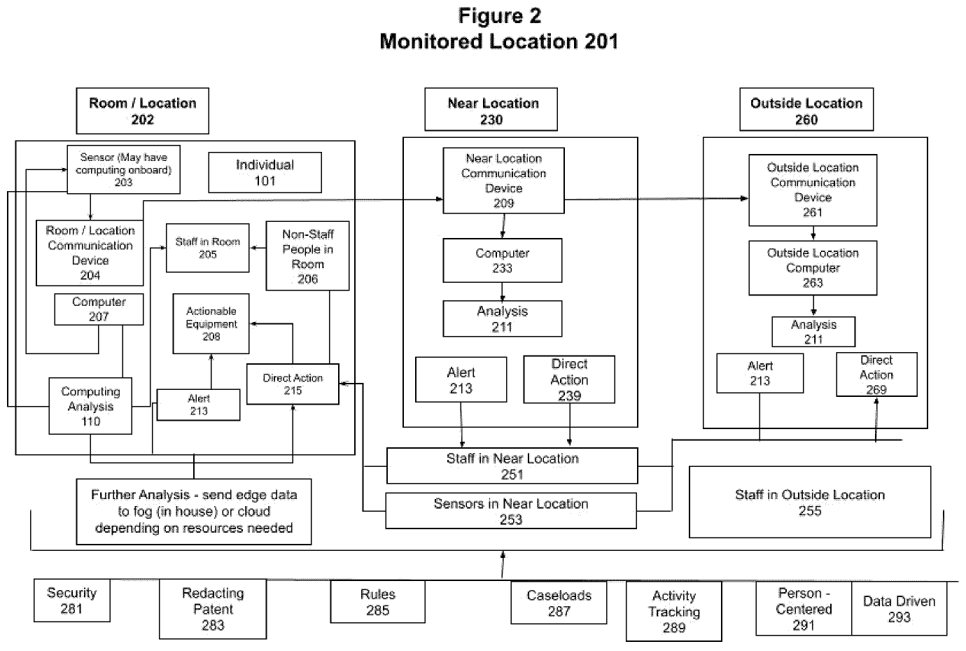

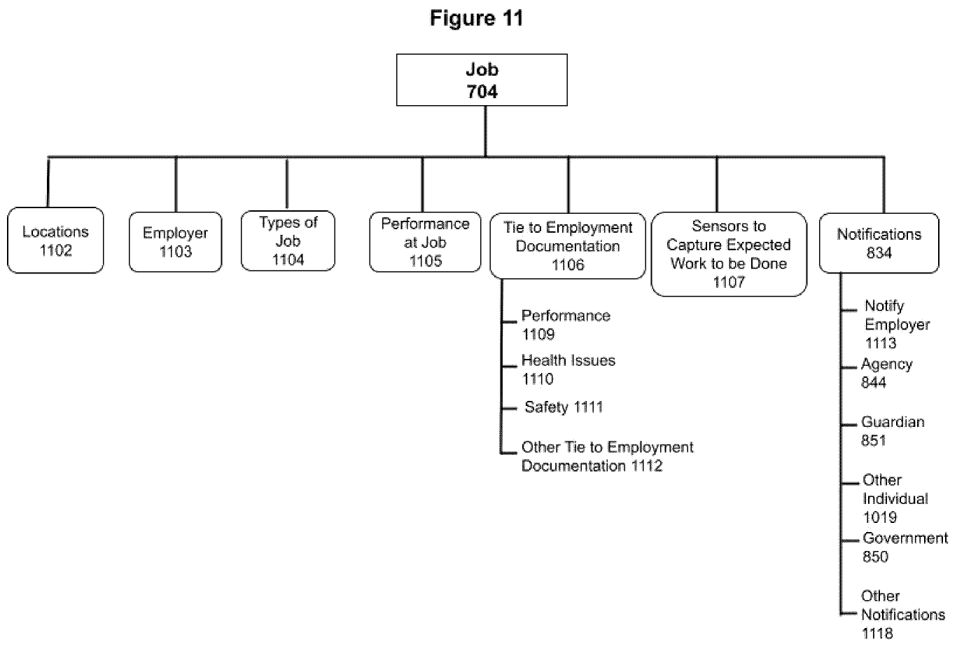

Computing Analysis 110 could be done in a remote off-site date center. One item which may be significant is the requirements for communications, electricity, and other connectivity between the sensor in the field and the computing device. The system could have a Smart Socket Device 141 which lets the system know that key network components such electricity, Internet, and communication network(s) including telephone service, are active, such as those described in the '316 Patent (and the '074 Publication at Paras. 0037-0041 and FIGS. 1-2 for basic function, and 0054-0065 and FIGS. 11-16 for specifics of an exemplary device).

The Computing Analysis 110 could be performed on a regular time schedule, as information is received or on other forms of schedule. Computing Analysis 110 could determine that a Plan of Action 112 is required or suggested. These plans of action could include notifying staff, documenting something in a record, activating a device to change something in the local environment, deactivating a device in a local environment, or other actions. These actions could have various levels of urgency. There could be actions with health and safety considerations or with concerns about preventing abuse and neglect. There also could be considerations about improving the quality of life for an individual such as adjusting a toaster because the individual might prefer their bread lightly toasted rather than heavily toasted and hot.

As a Plan of Action 112 is determined the Plan is Communicated 113 to one or more combinations of a Person 114, Device 115, or other means to implement the plan. For example if it is determined that an Individual 101 in a car is not seat-belted in properly the system could alert a Person 114 such as Staff 119 (or a guardian) who is in or near the vehicle that this preferably could or should be done or there could be a Device 115 such as an automatic seatbelt machine that would safely strap in the individual.

The system could appropriately document the Computing Analysis 110 performed, the Plan of Action 112 suggested and how the Plan is Communicated 113 in a secure documentation system based on appropriate roles and caseloads.

Just sending information about a proposed Plan of Action 112 may not be enough. An objective is to make sure that these plans have been implemented. So, the system could Track Information Received 131 by Person 114 or Device 115 or Track Actions Taken 133. If the information is not confirmed as received or confirmation of Track Actions Taken 133 then there could be a new Computing Analysis 110 performed factoring in this new information. At this point, for example, there could be a determination that a device is not working. For example if there is an Individual 101 in a Kitchen who is trying to access a locked kitchen utensil drawer in a smart kitchen with devices to lock and unlock based on certain predetermined conditions and a Plan of Action 112 was communicated to a locking Device 115 to unlock a drawer for access there could be a new notice to alert a Person 114 who might be asleep or working in another room in the building to assist in unlocking the device. There could also be a situation where an individual is living alone and the system could determine that a device is not working and might automatically call an electrician, plumber, or other professional. There could also be a need to contact social services or emergency first responders.

The system goes beyond the simple contact closures and motion sensors used in conventional alarm/notification systems. With this system, artificial intelligence Sensors 121 embedded in cameras or devices watching over an area may process the visual information that arrives through the camera lens and transform it into textual descriptions of what the camera is observing. This preserves privacy and allows the system to place cameras in places where they normally could not be deployed (e.g., bedrooms, bathrooms).

In addition, the Al processing may disambiguate what is “seen” by the camera; the textual descriptions can be used in complicated logic statements that indicate when an alarm preferably could or should be sent, or when remediation is necessary. For example,
“IF it is between the hours of 9 pm and 8 am AND
   A human individual entered the bathroom in the last 5 minutes AND
   There are lights on in the bathroom AND
   There is a person in the bathroom that has not moved in more than 1 minute
THEN
   Send an alert to a caregiver that the individual in Room X is likely to need assistance.”

The Sensors, Cameras and other physical devices can be combined with access to person-centered data about an individual which may include information on goals, outcomes, and historical information on their activities. In addition, the system can integrate into the analysis Public Information 125 from websites, social media, and other locations.

The system can integrate all of the sensors (smoke, fire, motion, choking, fall, apnea, aggression, self-injurious behavior, in-out of bed, heart rate anomaly, etc.) into a comprehensive system that manages alerts through one modality instead of having each sensor send its own alerts.

The system is designed to do more than just alert caregivers or staff to provide assistance. The system can notify Staff 119 or also notify Devices 115 to take actions based on analysis of what is happening.

Different people need different types of assistance or support. This assistance can vary based on the time of day and function they are serving. For example, if someone is a patient in a hospital there could be a variety of staff who are on duty to see a patient checked in and receiving services. If someone works in a supermarket doing some sort of supported employment there could be multiple other people working in that store. If someone were at some sort of community center for elderly or childcare or other large numbers of people during a day there could be multiple staff or helpers or volunteers on duty.

Where there can be a challenge is if someone is receiving support or care that is dependent on one individual. If that staff member were to die or become incapacitated while they were the only person providing support or monitoring, with a system to recognize this and notify the next staff or support person it could be of assistance to the individual who needed help as well as possibly provide lifesaving support to a sick or injured staff or support person.

One way that a system could determine that an individual was sick or died is through biometrics on the staff member. The Staff 119 could be monitored or tracked, for example, through a watch, ring, mattress, sleep mat, video camera, LiDAR, Hidden Camera, Facial IDs, Thermal Sensors, Bed Monitors, Sleep monitoring devices, Radar, robots, electronic pets, an infrared sensor, body cam or other type of monitoring system (see FIG. 13). In addition, having an application which at times causes the staff to actively state that they are on-duty or alert can serve as a double check for times when they are not available, which permits the system to be aware of the availability of the Staff 119 (Awareness of staff available in various locations 117). In those cases, there could need to be additional assistance sent to an Individual 101 receiving services as well as for the staff, support, or family who is supposed to be the first level of support at a given time.

FIG. 2 depicts an overview of a monitored location.

FIG. 2 depicts an overview of a monitored location. By definition, an Individual 101 is in Room/Location 202 that an Individual 101 is in. If the person moves to Near Location 230, for example from the bedroom to the kitchen, or from the bathroom to the dining room, whatever location that Individual 101 is in at any given time is the Room/Location 202 for that instant. If locations with regard to an Individual 101 who is being provided support are considered, there can be various people and Room/Location Communication Device 204 in the room the Individual 101 is in. The room can have other people in the room. This could include a Staff in Room 205, including multiple staff in the room. There could be other Non-Staff People in Room 206 who are people in the room who are friends, family, and other non-staff members.

For example:
a. Staff in Room 205 within a line of sight of the Individual 101
b. People nearby who can have a communication system which might not need to rely on civil or external services such as electricity, phone, or internet
c. Outside locations, which rely on traditional communication services such as electricity, phone, or internet.

The first room location which would provide line of sight to an Individual 101 traditionally meant that a staff member or family member or other person capable of providing support would actually be continually or periodically in a room checking on an Individual 101. This could mean that a staff member or person stepped into a room to determine if someone were sleeping, if someone were out of bed, if there was incontinence. This could mean that a staff member or person was physically checking on someone's attendance at work or school and their performance. This could mean that a staff member was continually watching someone at a playground, in a kitchen or other locations. There were several problems and challenges with this approach including the fact that there is a staffing shortage and high turnover of staff for service-related jobs, that staff might not be trained to know what to look for with certain individuals because of the change in staff, that staff could be waking an individual up when they show up in a room to check on bed awake or incontinence. The very act of checking if someone is sleeping could wake them up. Hospitals often have this situation where it is difficult for overnight patients to sleep given the noise of machines and people and frequent staff interruptions.

There could be a Room/Location 202 such as a bedroom where there are one or more sensors 203 (which include sensors 121) to monitor and provide information on what is going on. These sensors 203 could be communicating with a computer 207 in the room. This communication could be through Bluetooth, Wi-Fi, direct wire, or other means. The computer 207 in the room could then access information on the computer 207, through access to other computers and through the internet to do a Computing Analysis 110 of what is occurring. If there is a problem or something preferably could or should be changed, adjusted, or checked on, the computer could alert 213 either a member of staff in the room 205, staff outside the room, a device inside the room or a device outside the room to do something. The computer 207 could log and track this analysis 110. The computer 207 could also alert 213 a reporting system in a central location on this analysis 110.

For example, if one of the sensors tracked seizures and the computer 207 recognized that a situation required a staff member to take direct action 215, the computer 207 could alert 213 one or more staff members based on a predetermined schedule to come to the room. The staff member could acknowledge this request and start heading towards the room. The system could track the location of the staff member to confirm that they are heading towards the room. If a staff member does not respond within an appropriate time the computer 207 could contact another staff member to provide support. If required or if no staff member was available the computer might call 911 or emergency services.

Another example could be that a sensor 203 tracked that an Individual 101 was having trouble breathing or lack of oxygen through perhaps a pulse oximeter or heart rate or other type of monitor. In addition to contacting staff or emergency services the computer 207 might automatically activate an autonomous defibrillator or CPR machine which could be controlled without human interaction. This device could be prepositioned in the room or could be summoned from elsewhere in the facility or from another location. This system is not conditional on designing specific devices and sensors to monitor all situations which might occur. In society there are a large number of autonomous systems, devices, sensors 203, being created which can monitor, treat, support, and administer individuals or devices. An embodiment of this system is the integration of those devices when there is a need for determining access and support based on some or all of privacy and government regulations (such as HIPAA-type regulations), as well as security 281, caseloads 287 and rules 285 as described in the '370 Patent as referenced above, and other considerations.

Embodiments of this system include alerting or waking a staff member if there is a situation which changing a device cannot solve or improve. There is also the ability to alert 213 or wake a staff member if the electricity, internet, or phone are down. This can be accomplished by several means including a Wi-Fi alarm, a vibrating alarm, or another alarm. An objective of alerting the staff is to not disturb the individual 101 being supported. One method is using a Smart Socket Device 141 which lets the system know that key network components such electricity, Internet, and communication network(s) including telephone service, are active, such as those described in the '316 Patent (and the '074 Publication at Paras. 0037-0041 and FIGS. 1-2 for basic function, and 0054-0065 and FIGS. 11-16 for specifics of an exemplary device).

In the example, where the Staff is in a Near Location 230, several additional issues can arise. If all electricity, internet, and phones are working the Sensors in the Location with the individual 101 could either directly notify or alert 213 a staff member or it could be done through a computer 233 in the near location 230. Essentially if there were several locations being monitored, a more powerful computer 207 with additional access could be placed in a central location. This computer could be able to access and share information with computers in the location as well as with computers in outside locations. There could be communication between the Near Location Computer 233 and the Location Computer 207 even if communication, power, or internet with the Outside Location Computer 263 was down. The computers could have backup power, location-based Wi-Fi, Bluetooth, or other means of local communication. A goal of the sensors 203 and computers is to both maximize positive outcomes but also minimize negative outcomes for individuals. In particular there could be minimum targeted thresholds of safety for a given individual 101. There could be situations where based on road closures, outside events, information accessed from the internet certain devices in a location could lock certain doors and cabinets for example.

Outside Locations 260 could be locations which are accessed through the cloud, mobile phones, internet, Wi-Fi, and require electricity, internet, or other forms of technology or assistance to reach Outside Location Computers 263, Outside Location Communication Device 261 or Staff in Outside Location 255. One difference between a Near Location 230 and an Outside Location 260 is the level of technology required to access staff or sensors. So Staff in a Near Location 251 would not have line of sign to an individual 101 but a Room/Location Communication Device 204 could communicate with the Near Location Communication Device 231 with a reduction or elimination of technology or services including one or more of loss of electricity, loss of internet, computer, router, wireline phone note available, physical devices, Wi-Fi, radio part of network connection, networks, DNS failure, network failure, power failure, generator failure, cellular phones not available, government action to create any condition to not permit access to the internet or electricity, civil unrest, or other conditions.

Note that even systems which rely on the internet and electricity can be designed to use battery power in a short term. Or a cell phone or device can send notices or alerts if external access is not available.

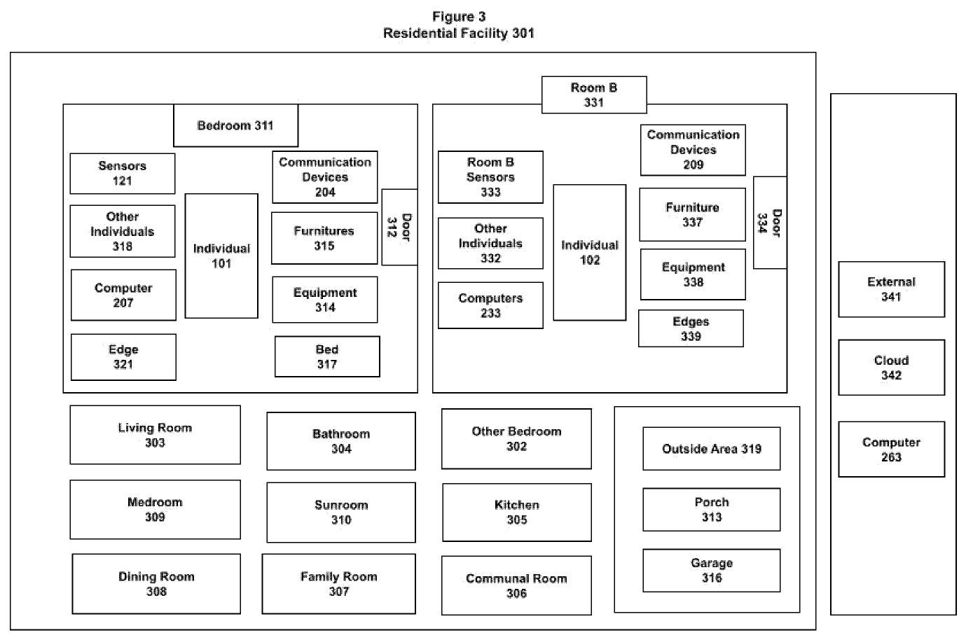

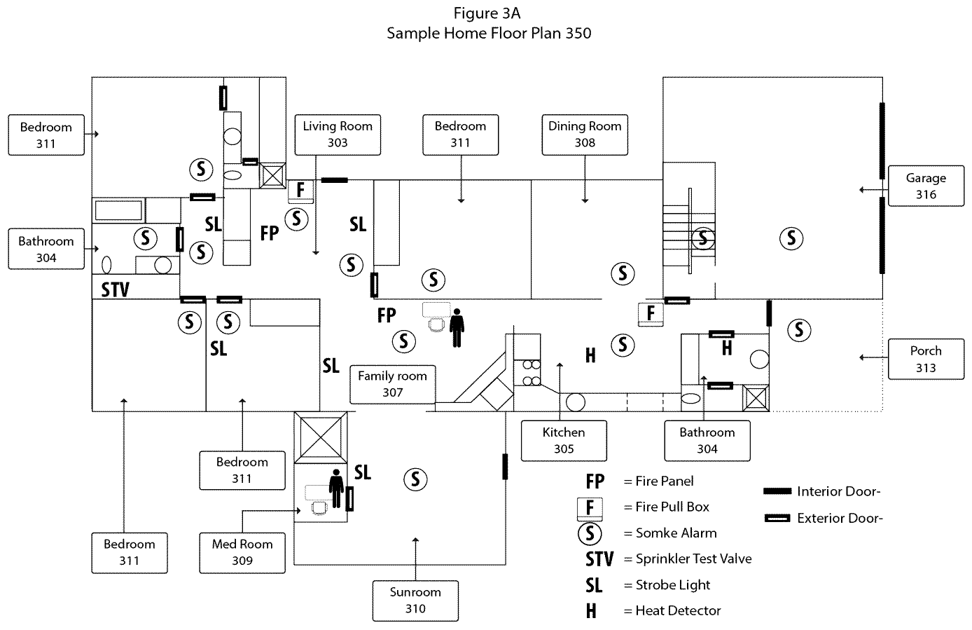

FIG. 3 depicts a residential facility.

FIG. 3 is a block diagram of a Residential Facility 301. A common location that Individuals 101 might need privacy, independence and be needed for continued access to information from devices is when in a Residential Facility 301. A Residential Facility 301 can be anywhere people live from a one-person facility to a large congregate care location where many people live.

Residential Facility 301 can have bedroom 311, living room 303, medroom (i.e., medication room) 309, dining room 308, bathroom 304, sunroom 310, family room 307, other bedroom 302, kitchen 305, communal room 306, porch 313, garage 316.

A bedroom 311 might have sensors 121 to monitor individuals 101 and other individuals 318, and process the data through communication devices 204 and store them in a computer 207. There can be other equipment 314 such as furniture 315, door 312, edge 321. In Room B 311, there might be room b sensors 333 monitoring individuals 102 and other individuals 332 through communication devices 209.

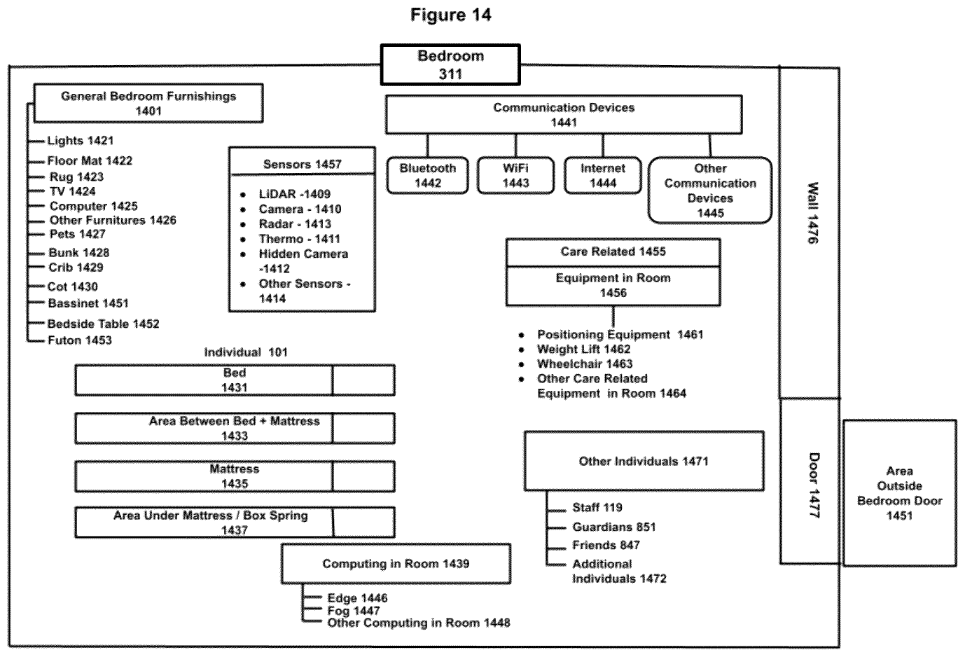

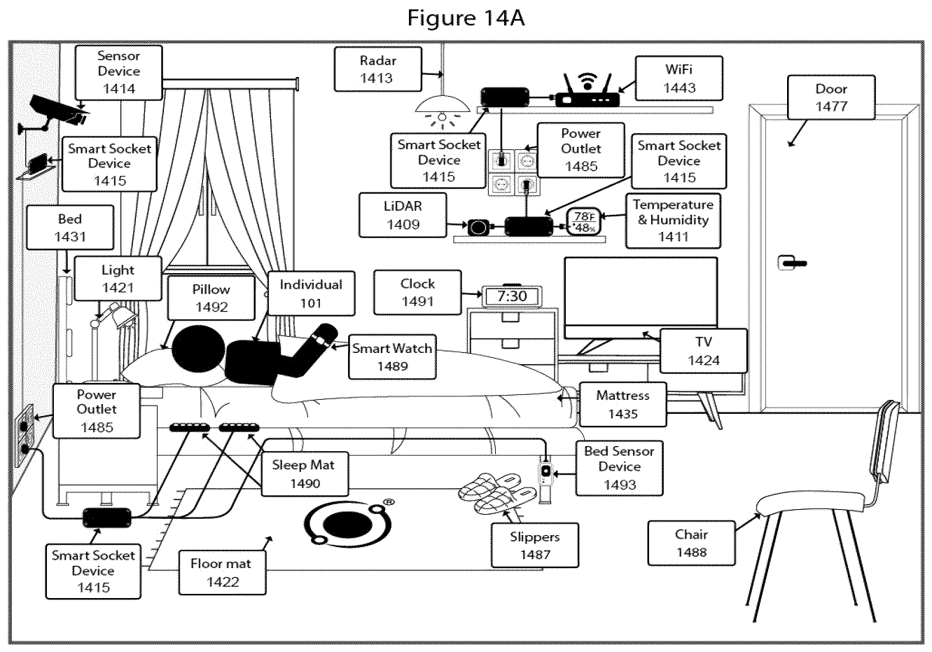

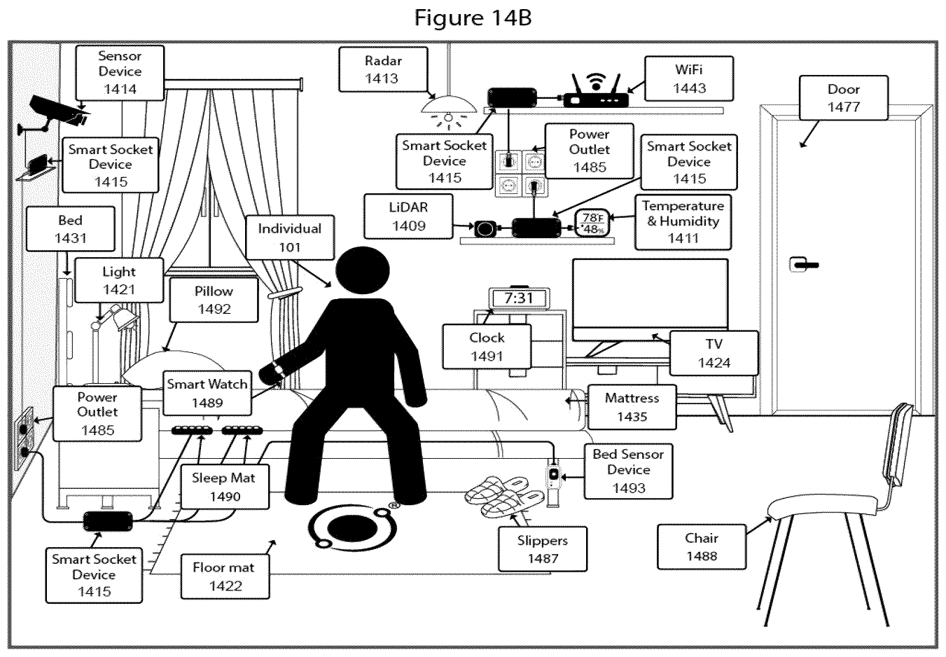

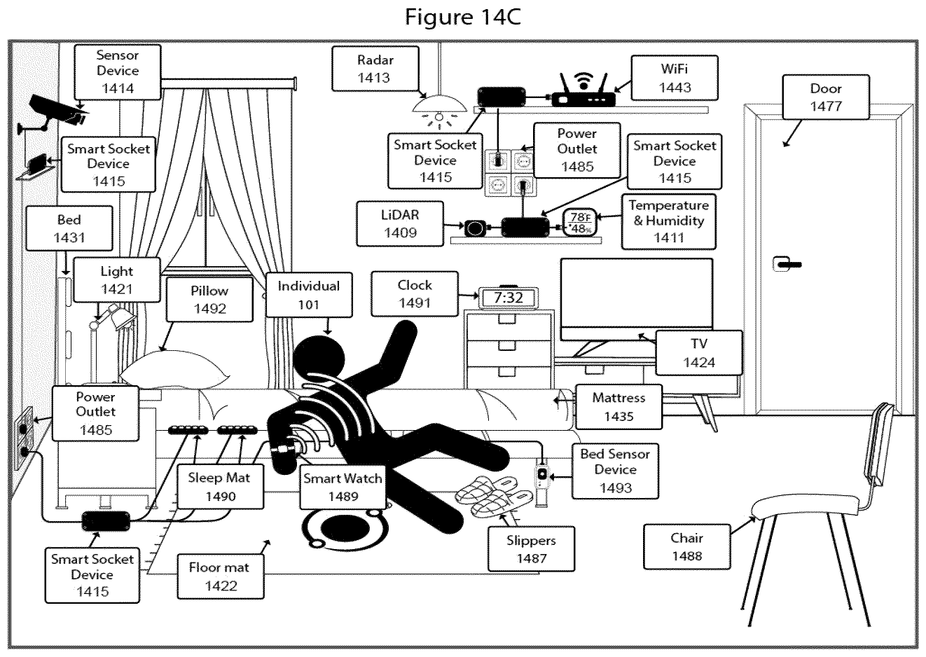

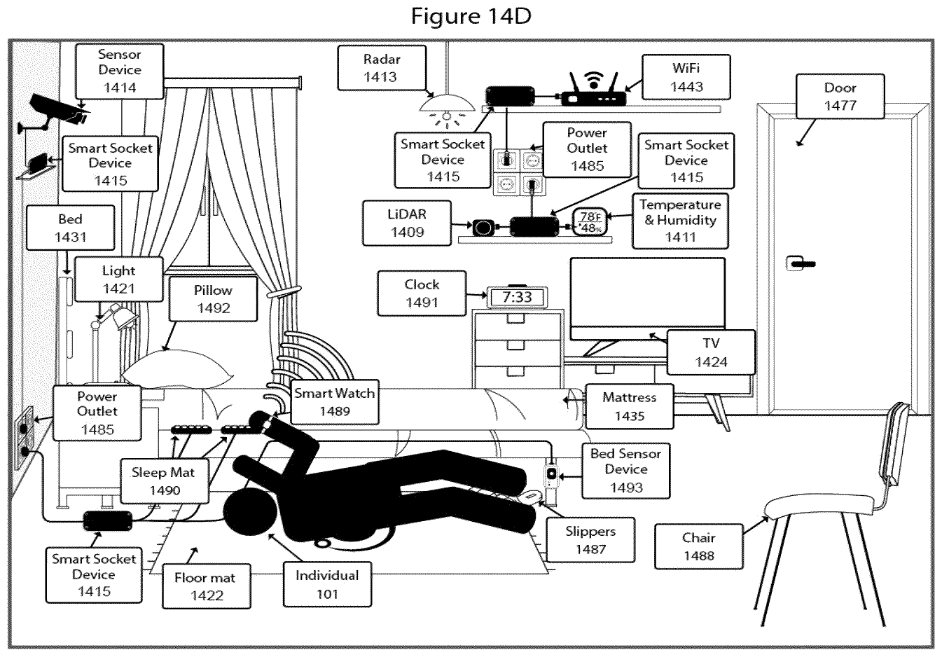

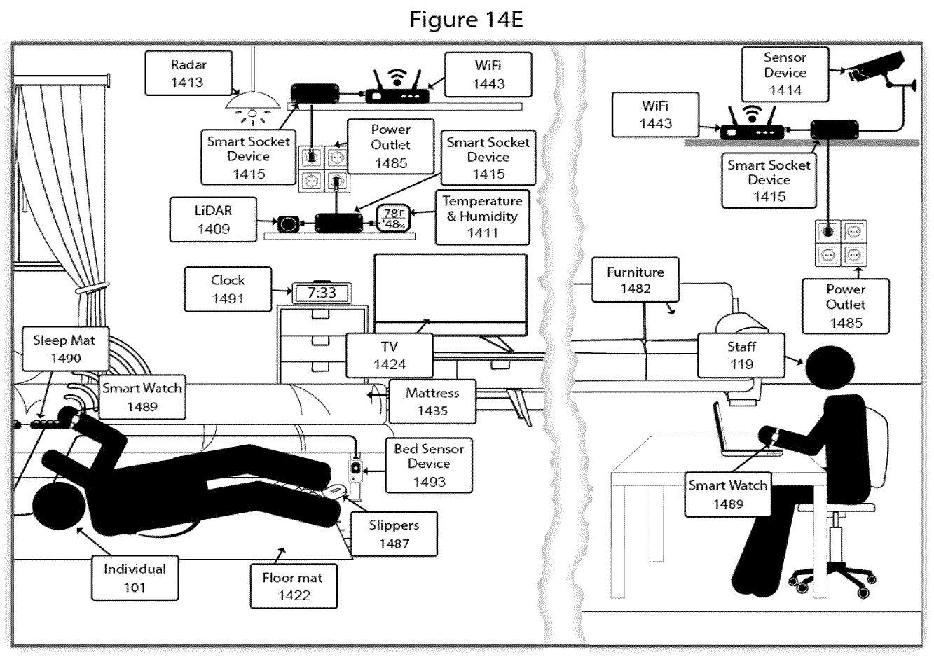

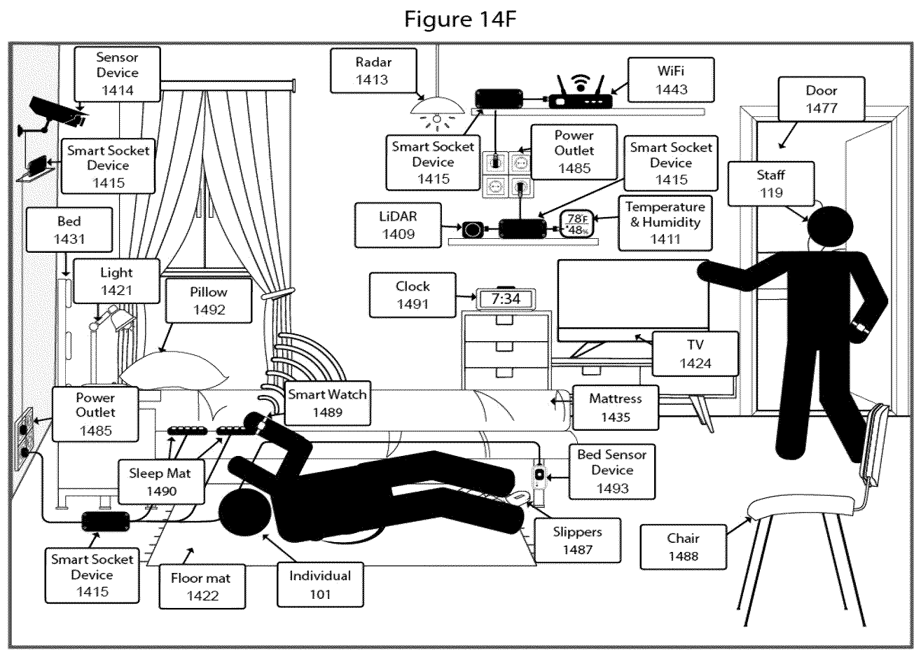

Outside of the bedroom 311 and room B 331 of the Residential Facility 301 there can be other elements such as external 341, cloud 342 and computer 263. Many incidents can happen in this residential facility for example an individual 101 can fall into the floor of the bedroom 311, and can get injured, which will be depicted in detail in FIG. 14C.

FIG. 3A depicts a sample home floor plan.

FIG. 3A shows a Sample Home Floor Plan 350 for a Residential Facility 301 which is an example of a purpose-built facility for multiple individuals receiving 24×7 support. In this example there are rooms including four Bedrooms 311, three Bathrooms 304, Garage 316, Porch 313, Dining Room 308, Living Room 303, Kitchen 305, and Sunroom 310. In addition, given the 24×7 nature of the space there could be a Med Room 309. At night while an Individual 101 is sleeping, the Staff 119 could be in one of these other locations and would either need to enter a room to check for falls, in/out of bed, incontinence or other situations or would need to receive information where they are elsewhere in the facility. Without some sort of monitoring system staff would have to go into each room to observe and analyze what is occurring. There could be instances where Staff 119 would sit either outside Bedroom 311 or even inside a Bedroom 311 to observe what is occurring.

Communication between the various rooms can require Wi-Fi, electricity, Bluetooth, battery power or reliance on other methods of transmitting information. There is a need for staff to be aware when these forms of communication are broken.

There could be an example where the staff is sleeping and living in one of the bedrooms 311 in which case the need for awareness that the means of communication is broken is more significant.

As illustrated in FIG. 3, locations in a Residential Facility 301 could include a Bedroom 311, Bathroom 304, Kitchen 325, Garage 327, Communal Room 328, Dining Room 308, Other Room B 331, and Outside Area 319. In a Group Home or Congregate Living Home with staff there could be a Staff Sleeping Room where the staff could be asleep or awake in that room while an Individual 101 is sleeping in their Bedroom 311, awake in their Bedroom 311, using the Bathroom 304 or engaged in other activities in the Residential Facility 301.

A Bedroom 311 could normally be expected to have a Bed 317, Furniture 315, Door 312, and other furnishings. In addition, there could be Computers 207, Sensors 121, Edge 321, and Communication Devices 204 added to the room as part of an effort to communicate information about what is happening inside the room to people outside the room and then receive instructions from outside the room for things that preferably could or should happen inside the room. There could be an effort to change activities which are occurring, might be occurring as well as documenting what is happening for regulatory, funding, compliance, support, improved outcomes, or other reasons.

Individuals can live in many types of locations; one type of living situation is a home with other individuals with a similar condition.

FIG. 3A shows a detailed blueprint of a group home, a Residential Facility 301, tailored for individuals with disabilities, which could integrate a residence for support staff as well. This layout is structured in a way so that it can cater to the safety and ease of navigation for residents.

The home can include multiple Bedrooms 311 (one of which could be for staff), Bathrooms 304, a Kitchen 305, Dining Room 308, Living Room 303, Family Room 307, Sunroom 310, Medical Room 309, and an associated Porch 313 and Garage 316. These rooms can be equipped with specific safety and monitoring devices such as smoke and fire alarms, heat detectors, fire pull boxes, fire panels, and others, which can be strategically positioned throughout the premises to maximize safety protocols. Strobe lights can be associated with fire alarms that provide visual alerts such as horn alarms and strobe notices to assist those with hearing impairments. The rooms can also incorporate features for those with visual impairments that can enhance environmental visibility and navigation. These include high-contrast color schemes in rooms and hallways, tactile indicators on flooring and walls, and Braille labels on room entrances and appliance controls and others. For mobility impairments, the residence can be equipped with wide doorways, ramps for easier access, grab bars in bathrooms and corridors, and others, to aid movement.

Additionally, the inclusion of a fire panel and sprinkler test valves can ensure that a comprehensive response system is in place to manage fire-related emergencies effectively.

These systems can be interconnected with both interior and exterior pathways, marked distinctly to facilitate quick evacuation or access by emergency personnel. Moreover, monitoring and sensor devices such as LiDARs, Cameras, body cam, facial ID, hidden cameras, Thermal Sensors, Radar, infrared cameras, pet animals, robots, and other devices (see FIG. 13) can be strategically placed in every room of the group home so that staffs can quickly be alerted in case any incidents might occur.

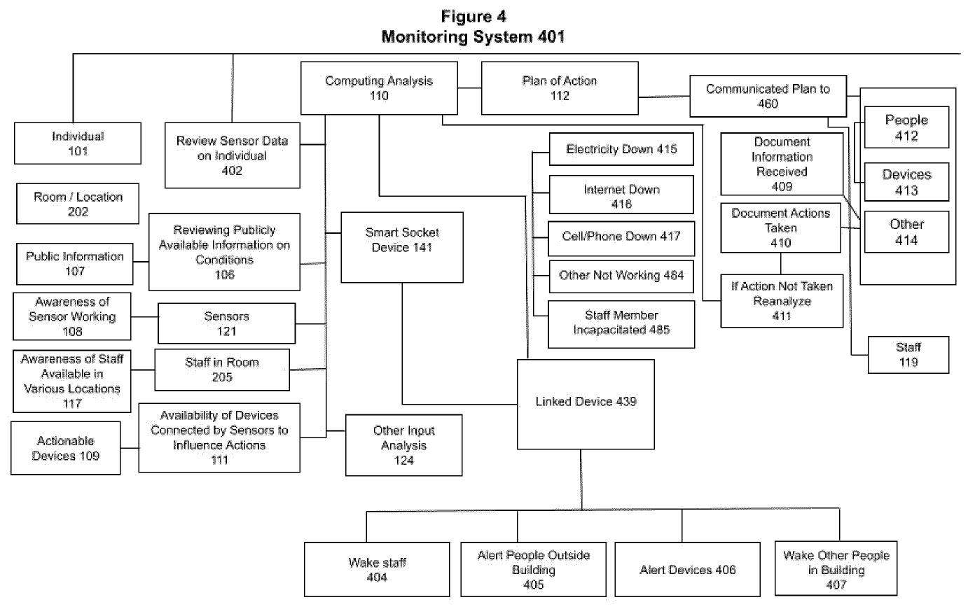

FIG. 4 depicts a monitoring system.

FIG. 4 shows an example of a Monitoring System 401. Individual 101 is in a Monitored Location 201. In a normal scenario when all information flows are working there could be a Sensor 203 in Room/Location 202. That sensor could either have computing capabilities on the sensor device to Review Sensor Data on Individual 410. Sensor 203 or Computer 207 in the Room/Location 202 could perform Computing Analysis 110 of what is happening. For example, sensors 121 could determine from information that everything was essentially fine and that no changes in action needed to be taken. In this example, the Individual 101 was sleeping normally with a heart rate within normal parameters and other biometrics within a normal and appropriate level. The sensor 121 and computer could determine that the Staff in Room 205 is awake and acting appropriately. Sensor 203 could determine that other monitoring devices in the room were all in a normal non-concerning state. These other devices could include, for example heat, air-conditioning, HVAC, water flows, sump pumps, electricity, burglar alarms, door alarms, lights, internet, television, electricity, and other types of information which might affect safety or other concerns regarding negative outcomes or the ability to have positive outcomes for an individual. In this example for example, the output of Computing Analysis 110 might be that everything is ok and no alerts or notifications or changes are needed. In that case the Plan of Action 440 could be to do nothing. There could be a Communication Plan 460 communicated to the Staff in Room 205 that everything is normal or there might not be a need to communicate that to the staff to avoid overwhelming them with too much data or information. There could be a desire or need for regulatory or documentation to Track Information Received 463 periodically in order to have a record of what was occurring.

Another example of systems working but actually needing to notify staff and take action could be that Individual 101 fell on the floor in the middle of the night. In this instance, the Sensor 121 could have received data indicating that it appears the individual 101 might have fallen. It is actually possible that an individual 101 could have been on the floor without falling and in no danger if, for example, they dropped their glasses or a pen on the floor. So, the system could have the ability to make inferences and determine what it believed happened. In the situation where the individual 101 fell the Sensor 203 would Review Sensor Data on Individual 401 and perform a Computing Analysis 110 and determine someone had likely or possibly fallen. The Plan of Action 402 might be to Communicate Plan 460 to the Staff in Room 205 or a Staff Nearby 251 via a cell phone, internet message, or other mechanism for the Plan of Action 440 to check on the individual. The notice would be in an appropriate manner given HIPAA-type regulations, PII, security, and privacy of information based on the mode of communication. The system would Document Information Received 463 and Document Actions Taken 465. The sensors 203 and computers 207 and 233 would monitor to make sure that the suggested actions were taken and implemented. The action could include going to the Individual 101 on the floor and seeing that they are okay, asking why they are on the floor, were they hurt, did they need assistance getting up, is there a need to contact emergency services, and other possible interactions.

There could be a situation where there was no Staff 119 in the room. There could have been Actionable Devices 209 such as robots, electronic pets, electronic devices, adjustments to the environment including alarms, adjustments to heating, cooling, and noise levels. There could be situations where individuals 101 are living alone with no staff 119 and their assistance comes from devices. Perhaps a robotic dog would be sent to bring them food or water, lights could be turned on so they can see what is going on in the room, an electronic harness might be deployed from the ceiling to help pick the person up and carry them back to their bed, an automatic incontinence management system might help empty their diaper, clean their body and put on a new diaper, or one of many other scenarios which already exist or could be developed in the future. This invention is designed to work with future to-be-invented technologies as well as existing products and applications.

In the preceding paragraphs the assumption is that equipment, communications modalities, electricity, and other infrastructure were operating normally. But what happens in situations where there are problems? What happens if the Electricity Down 481, Internet Down 482, Cell Phone Down 483, Other Not Working 484, Staff Member Incapacitated 485. By down or not working we mean those tools are not functioning and not available for normal operations of an information and communication flow.

In the example where an individual 101 was out of Bed at night and the overall system design was relying on some sort of notification to a staff member to come to help or action to be taken by an automated device, but electricity and internet were not available there is a need for a new device or process. Embodiments of this system provide for a Smart Socket Device 430 as described in the '316 Patent as referenced above. This device will notice when the sensors 203 and devices are not able to complete their required tasks and will then be able to know what regular tools are down (electricity, internet, cell phone) and then take actions as possible to either notify staff or take other action.

In a situation where the staff is nearby or in the same building, the staff could have a Linked Device 439 which receives information on a regular basis from the Smart Socket Device 430 stating that systems are ok. If Linked Device 439 does not receive a ping or other notification that systems are ok and working, there would be an indication to the staff member that systems were not working properly (Electricity Down 481, Internet Down 482, Cell/Phone Down 483) and they preferably could or should return to a manual type monitoring and support approach. The agency might have prepared paper-based guidelines to review and refer to in this situation.

In a situation where the staff is further away, they could have the same Linked Device 439, but it might take longer for staff to get to Individual 101 to provide assistance.

There could be a situation where an agency loses the ability to communicate with staff team members due to Electricity Down 481, Internet Down 482, and Cell Phone Down 483. In those instances, there would need to be a network of messages for information not received by key staff or management so places would understand actions which might need to be taken based on the limited information available.

There could be a need to have access to public information 107 to help in the decision-making process. This Public Information 107 could arrive via internet, GPS, satellite, or other source and deliver publicly available information from sources including weather apps, traffic apps, news apps, bulletin boards, crowdsourcing information, chat rooms, or other non-proprietary sources of information.

The Computing Analysis 110 could include reviewing 106 this public information 107 in determining Plans of Action 112. For example, if a regional manager received notice from Linked Device 439 that was in their presence that they had not received information from Smart Socket Device 141 stating that the system was working properly, an analysis which included access for example to the electric companies grid showing that electricity was depicts a circle of support down at a specific house location, combined with information from a wireless mobile phone carrier that cellphones were down in that same part of town, combined with a weather website showing there were thunderstorms in the neighborhood combined with a GPS map service showing that traffic was normal in that neighborhood would allow the system or an agency manager to decide to send someone to that location to make sure staff is awake and not expecting to rely on automated information. However, if this same information was across 5 locations then the manager might have to send an-off duty supervisor of these locations.

Embodiments of this system provide for attempting to use various telephone, mobile phone, internet, and other tracking means of reaching a main office. For example, if there are known GPS tracking devices in a car which might work through a different network, the on location Smart Socket Device could know that an attempt to activate a way of sending a message that an on-site staff was awake and functioning to avoid the cost and time of sending another staff to the location.

Embodiments of this system provide for additional means of communications such as small-scale satellite dishes which might provide limited means of messaging.

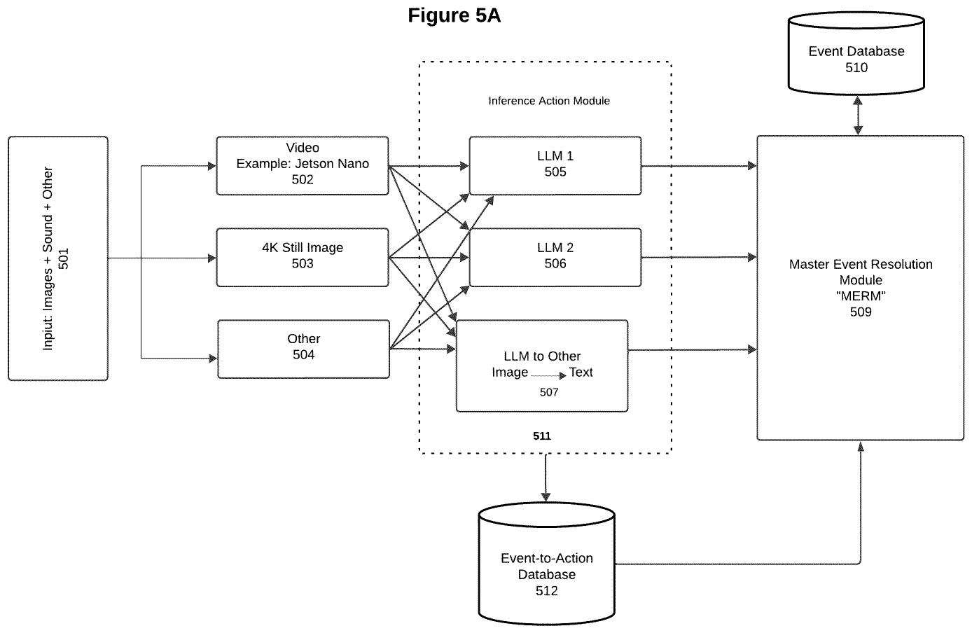

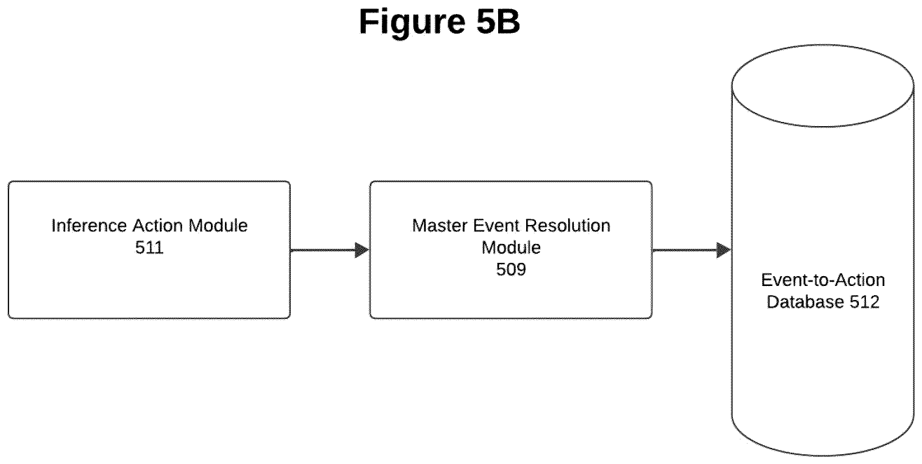

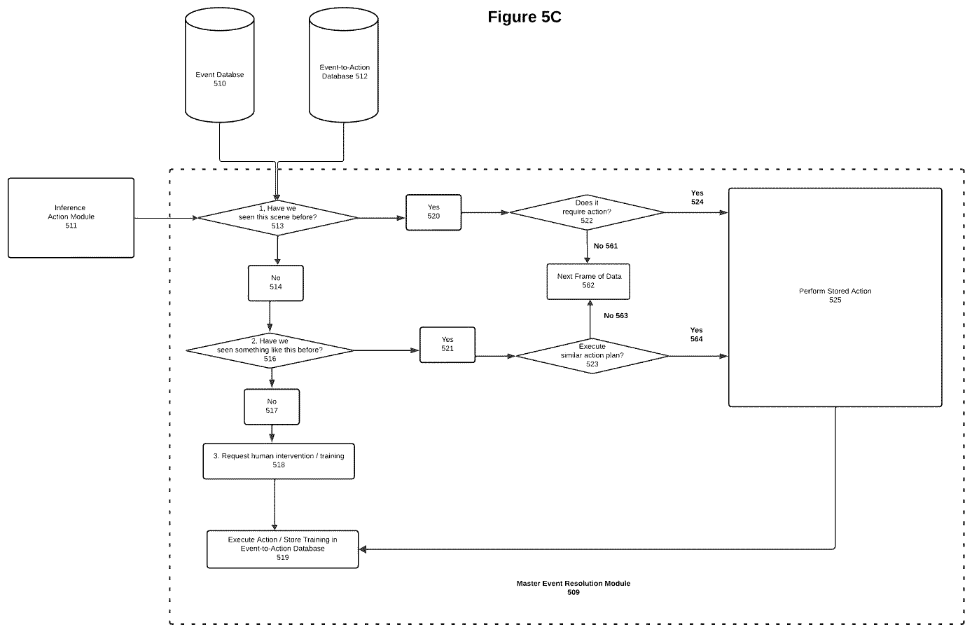

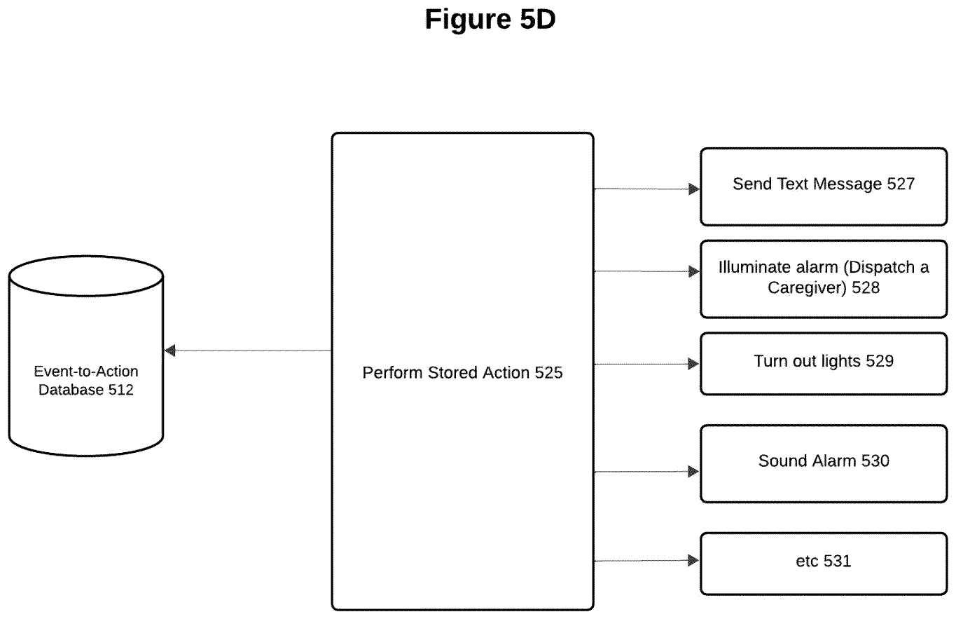

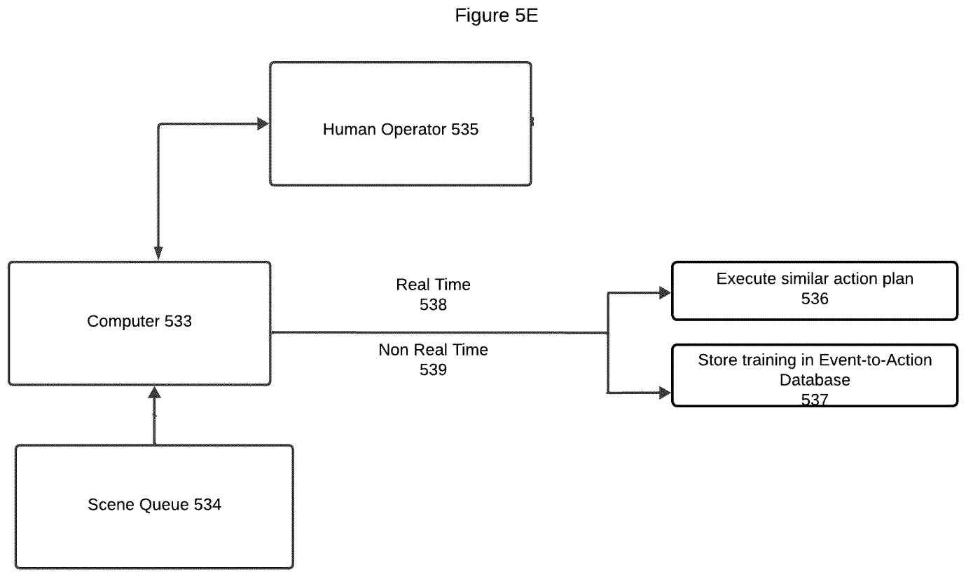

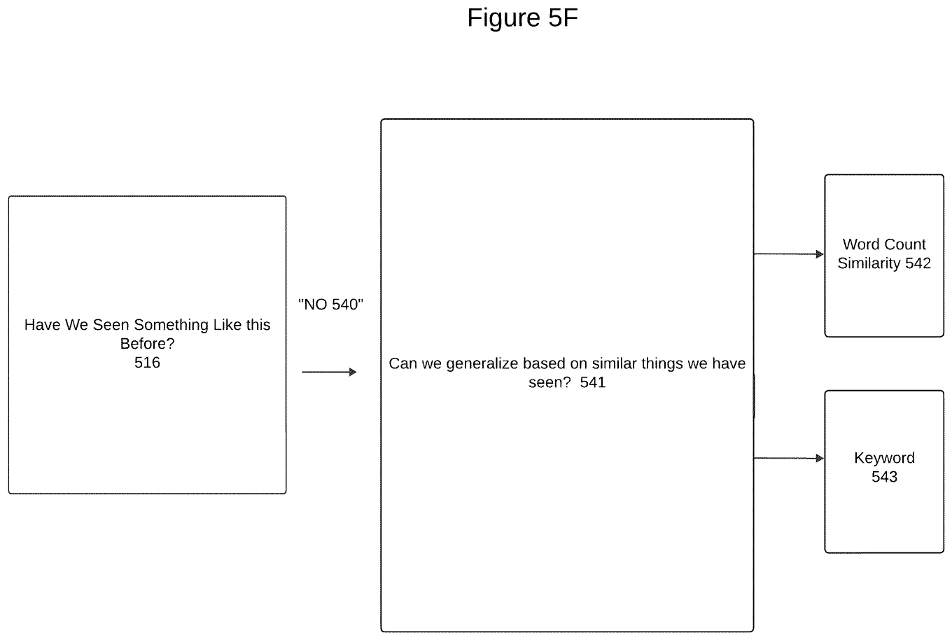

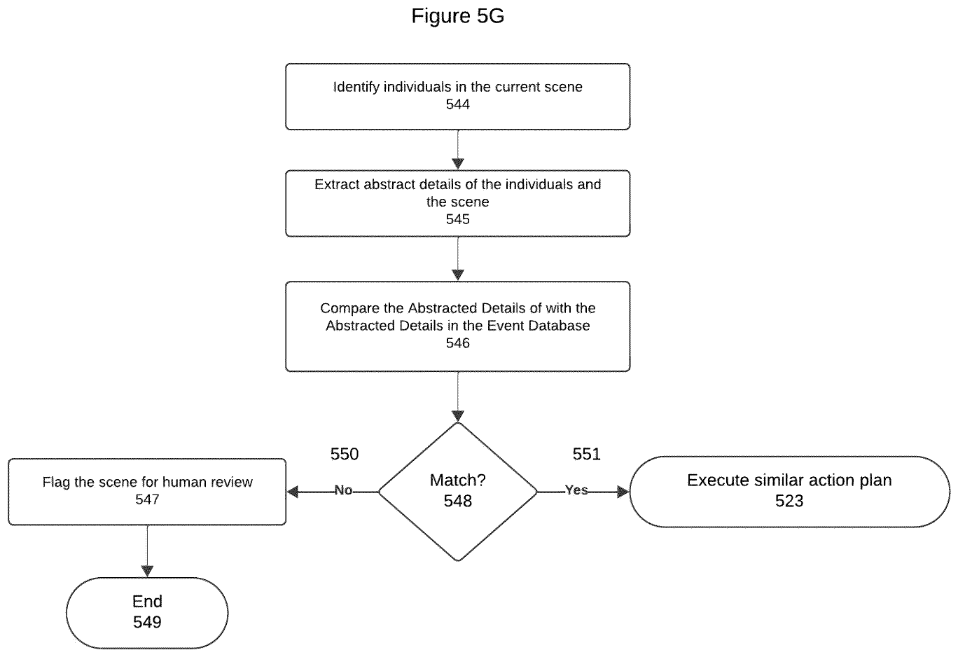

FIGS. 5A-5G illustrate a preferred embodiment of a Computing Analysis 110.

FIG. 5A depicts the images, videos, sounds, and other data processed into texts by the Inference Action Module.

In FIG. 5A, on the left there is an input 501, which could be the combination of different things that came from sensors like image, sound, video etc. This also might be the room temperature or lights in the room as well. These inputs will go into different sensors which are Video, Example: Jetson Nano 502, 4K still image 503, other 504. After that, the Sensors can talk to the different large language models, like LLM 1 505, LLM 2 506, LLM to other 507, referred to as Inference Action Module 511. Inference Action Module 511 creates textual descriptions of what the sensor believes is happening in the room. Jetson Nano 502 has a visual image that's passed to Inference Action Module 511. Inference Action Module 511 takes these multiple event descriptions and resolves them into a unified understanding of what's happening in the scene. It combines the textual descriptions of the different sensors, and converts that into a single text description. These descriptions may be synchronized by frame, for example, if it's a video, then this will be a frame per second rate. Inference Action Module 511 sends that single text description to the Master Event Resolution Module 509. Data from Inference Action Module 511 may be stored in Event-to-Action Database 512.

FIG. 5B depicts the Inference Action Module going to the Master Event Resolution Module and the Event-to-Action Database.

FIG. 5B shows Master Event Resolution Module 509, takes the text description from Inference Action Module 511, determines an action, and stores the data in an Event-to-Action Database 512. Event-to-Action Database 512 stores date, time, persons that are in view, or in the previous description where the identifiers are known. They could be part of that database or part of the database entry.

FIG. 5C depicts a workflow of the Master Event Resolution Module.

FIG. 5C illustrates that Master Event Resolution Module 509 takes text description data from the Inference Action Module 511 and then consults with the Event Database 510. In the Master Event Resolution Module 509 there are some actionable items and scenarios that might happen. The Master Event Resolution Module 509 looks at the summary from the Event Database 510 and detects what's happening in the room, and based on that, within the Master Event Resolution Module 509, a number of questions are being asked. The first question is, Have we seen this scene before? 513. Which could mean that something that is not necessarily seen with our eyes but, has something occurred similar to this before? This preferably requires a comparison of the text description from the Inference Action Module 511 with the information in Event Database 510.

If the answer to 513 is Yes 520, then we say, Does it Require Action? 522 If the answer to 522 is Yes 524, then we Perform Stored Action 525. If the answer to 522 is No 561, then the system moves on the Next Frame of Data 562, beginning the process over again.

If the answer to 513 is No 514, that we haven't seen something like this before, then there will be a new question, Have we Seen Something Like this Before? 516.

And if the answer to 516 is No 517, then we need to Request Human Intervention/Training 518 with the system so that the system will know what to do and what to do next time it sees something like it. At this point, the human preferably trains the system that this is the sort of action that you need to do when you see this event. This process is Reinforcement Learning from Human Feedback (“RFHF”). The action input by the human is stored as training 519 in Event-to-Action Database 512.

If the answer to 516 is Yes 521, then we ask Is There a Similar Action Plan? 523 to the one that's known. If the answer to 523 is Yes 564, this will go to Perform Stored Action 525. If the answer to 523 is No 563, then the system moves on the Next Frame of Data 562, beginning the process over again.

Regardless of the answers to 513 and 516, and whether the action performed is in the Event Database 510 (i.e., Performed Stored Action 525) or newly added by human training, the summary and action are stored (519) in the Event-to-Action Database 512.

Although the preferred embodiment described herein refers to the Event Database 510 and the Event-to-Action Database 512 as separate databases, these could be combined into a single database having the function of these two databases.

FIG. 5D depicts performed stored actions creating different alerts.

FIG. 5D depicts that after the Perform Stored Action 525 is executed, that training input is stored in the Event-to-Action Database 512. If the system had never encountered something where one person punched another in the face, then we have to go down to where we requested human training. When the system requested human training, the human trainer could say, for example, the system preferably could or should send an alert such as Send Text Message 527, Illuminate alarm (Dispatch a Caregiver) 528, Turn out lights 529 (or turn them on), Sound Alarm 530, etc. 531 to someone. And the caregiver or another person enters the room to break up the fight before it gets worse. So that data is then stored back in the Event-to-Action Database 512.

Now, under Perform Stored Action 525, there are a number of high-level actions that can be taken, and then a number of specific actions that can be taken. Anything that can be thought of can be affected through an automated process in an Internet-of-Things-based device.

FIG. 5E depicts execution of similar action or stored training in the Event-to-Action Database.

In FIG. 5E, the descriptions of events that have happened or that are currently happening, that the system doesn't know and the Event-to-Action Database 512 has not been seen before in some form, so it doesn't know what to do and can't do anything automatically.

And so, it sends that description out into a Scene Queue 534, and the different scenes in the queue are there waiting for human intervention so that the Human Operator 535 can basically train the AI what it preferably could or should do when it sees this or a very similar scene, i.e., Reinforcement Learning from Human Feedback. That Scene Queue 534 offloaded one at a time to a Human Operator 535 who is working on a Computer 533 or another similar device. And the person describes the scene. And the human operator decides what to do with it and passes that information. If it is in Real Time 538 then that should be Execute Similar Action Plan 536. If it's Not real Time 539, the next step preferably could be to store the human training input, i.e., Store Training in Event-to-Action Database 537, so that when that scene occurs again, then the action can be taken with a high degree of automaticity.

FIG. 5F depicts using word counts similarity and keywords to identify an event.

FIG. 5F shows a process for determining the answer to the question: Have We Seen Something Like this Before 516? And as the answer was No 540. So, after that there will be another question: Can we generalize based on similar things we have seen 541? Then the module preferably could or should look through the Event-to-Action Database 512. And there are a number of ways that could be done. The first is through Word Count Similarity 542. Another might be Keyword 543.

FIG. 5G depicts a workflow for identifying an event by the Inference Action Module.

FIG. 5G is, based on the previous description, in boxes 513 and 516, the system preferably could or should Identify Individuals in the Current Scene 544. Exemplary methods and systems for identifying individuals that may be integrated with embodiments of this invention, including by facial recognition or by matching one of a face, eye color, and/or hair color, are disclosed in the '806 patent at least at: Col. 40, lines 33-48; Col. 44, lines 14-16; Col. 46, lines 23-48; and Col. 50, lines 22-62, which as noted above is incorporated by reference herein. Exemplary individual identification systems and methods that may be integrated with embodiments of this invention are also disclosed in the '354 application and are incorporated by reference herein. After detecting or Identifying the Individual the Inference Acton Module 511 could Extract abstract Details of the Individuals and the Scene 545. Then in the next step the system will Compare the Abstracted Details of with the Abstracted Details in the Event Database 546. If the details Match? 548 with the database and if the answer to 548 is Yes 551 then the system will Execute Similar Action Plan 523. If the answer to 548 is No 550 then the system will Flag the Scene for Human Review 547.

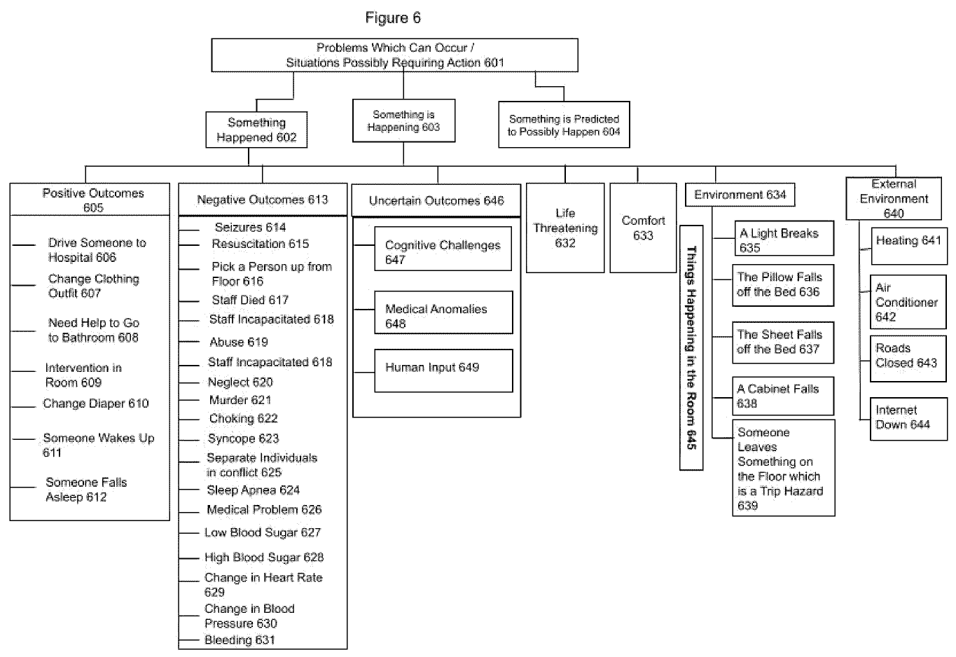

FIG. 6 depicts problems which can occur, and situations possibly requiring action.

FIG. 6 depicts problems or situations that might require intervention, illustrating potential actions that could address various circumstances. Something Happened 602, Something is Happening 603, and Something is Predicted to Possibly Happen 604 are classifications that describe temporal aspects of issues requiring attention. Immediate actions might be necessary to respond to events in progress or proactively to those anticipated.

Circumstances such as Life Threatening 632 situations involve critical events, including Choking 622, Murder 621, or severe medical conditions such as Seizures 614, Syncope 623, or Sleep Apnea 624. These might require urgent interventions, such as Resuscitation 615 or addressing significant injuries like Bleeding 631. Individuals might need support for medical concerns like Low Blood Sugar 627, High Blood Sugar 628, or changes in vital signs such as Change in Heart Rate 629 and Change in Blood Pressure 630.

Positive Outcomes 605 indicate desirable or beneficial scenarios, such as improvements in health or resolution of problems, while negative outcomes Negative Outcomes 613 reflect adverse situations that could require attention to prevent worsening conditions. A category of uncertain outcomes 646 could also capture situations where data analysis or human intervention is necessary to interpret ambiguous scenarios effectively.

Uncertain Outcomes 646 represent scenarios where the event or response is ambiguous, requiring deeper analysis or human intervention. These could range from relatively simple incidents, like A Light Break 635 or A Cabinet Fall 638, to more complex issues like behavioral changes or Medical anomalies 648. For individuals with cognitive challenges 647, uncertain outcomes 646 highlight how previous data can guide responses. For instance, an individual with cognitive insensitivity to pain might not visibly react to a burn, but reviewing past situations or live footage could alert caregivers to respond appropriately. Incorporating mechanisms like dropdown menus or human input 649 for data categorization allows the system to adapt and provide informed recommendations, even in unfamiliar situations, ensuring both immediate and future support is optimized.

Daily challenges such as personal care might also arise, like Change Clothing Outfit 607, Change Diaper 610, or providing assistance when a person Needs Help to Go to the Bathroom 608. Environmental mishaps, such as A Light Breaking 635, The Sheet Falling Off the Bed 637, or A Cabinet Falling 638, could require swift action to ensure safety.

Social or behavioral contexts, including Abuse 619, Neglect 620, or conflicts requiring intervention such as Separate Individuals in Conflict 625, might demand immediate attention. These could escalate into more serious conditions if unaddressed.

External Environmental 640 factors are essential as well. Issues such as Heating 641, Air Conditioner 642, or disruptions like Internet Down 644 and Roads Closed 643 might influence the functioning of the setting. Environment 634 factors can affect individuals differently depending on personal conditions and context. For example, temperature changes might discomfort some individuals while being critical for others with specific health conditions.

In emergencies, tasks such as Driving Someone to the Hospital 606, Picking Someone Up from the Floor 616, or assisting if Staff Are Incapacitated 618 or Staff Have Died 617 highlight the importance of adaptable response systems.

The intricate dynamics of interactions in various environments, Things Happening in the Room 645, and the external setting, External Environment 640, preferably require tailored interventions to support individuals in maintaining comfort and safety.

Addressing the scenarios described in FIG. 6 might involve a combination of monitoring, prediction, and prompt action. By clearly defining situations, tracking experiences through recorded data, and analyzing patterns, the system could provide more informed, proactive support and assistance while mitigating uncertainty in unfamiliar conditions.

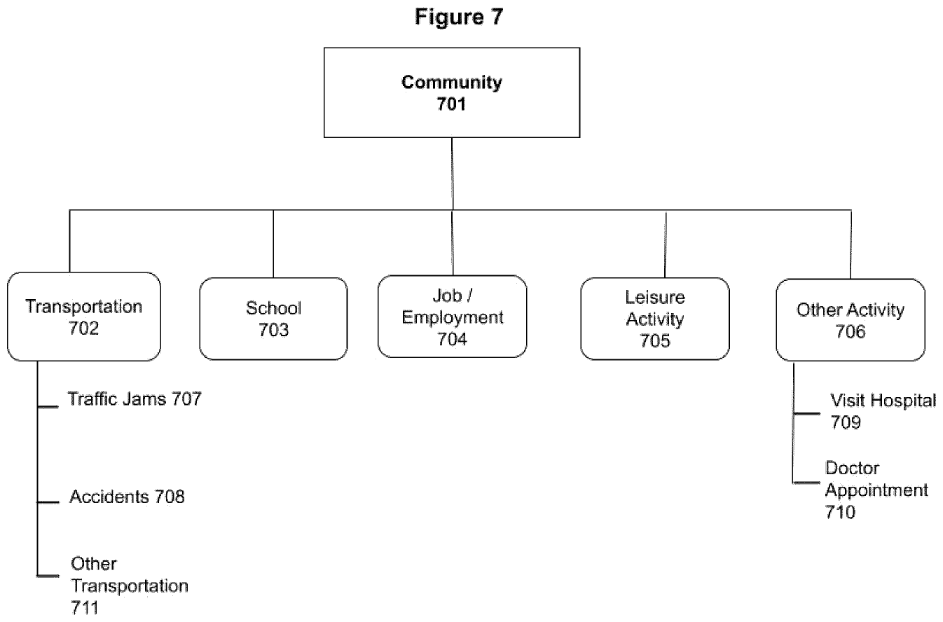

FIG. 7 depicts community locations which might utilize aspects of the invention.

FIG. 7 shows other Community 701 locations where an individual 101 could be, which might utilize aspects of this invention. An Individual 101 could be on modes of Transportation 702 including a bus, car, subway, airplane, boat, truck, or other vehicle. The vehicle could be a Traffic Accident 708, Traffic Jam 707, or have another incident. For example, if an individual were travelling in a bus which had sensor devices which indicated a potential problem a staff member could meet the bus prepared to deal with the situation.

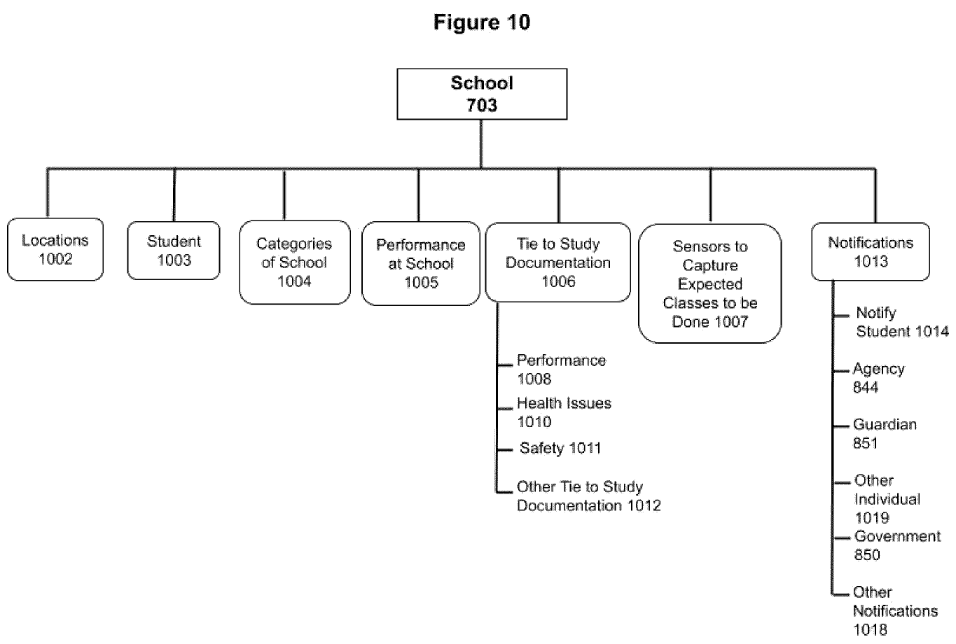

As individuals are looking for more independence and sensors and devices become more capable of providing data to assist this independent individual there could for example be less staff directly in line of staff supporting an individual. Examples of locations could include within Schools 703 at gyms, bathrooms, classrooms, hallways, libraries, meeting rooms, playground, and other locations (See FIG. 10). Another location could include employment and workforce locations 704 (See FIG. 11). For example, people might have had staff to assist in their independence to learn and perform jobs. There could be individuals with developmental disabilities or other disabilities or conditions who could have staff or systems for training or ongoing assistance for vocational training or vocational rehabilitation. As technology, sensors and devices can help individuals achieve independence there is a question of what happens when the technology stops working.

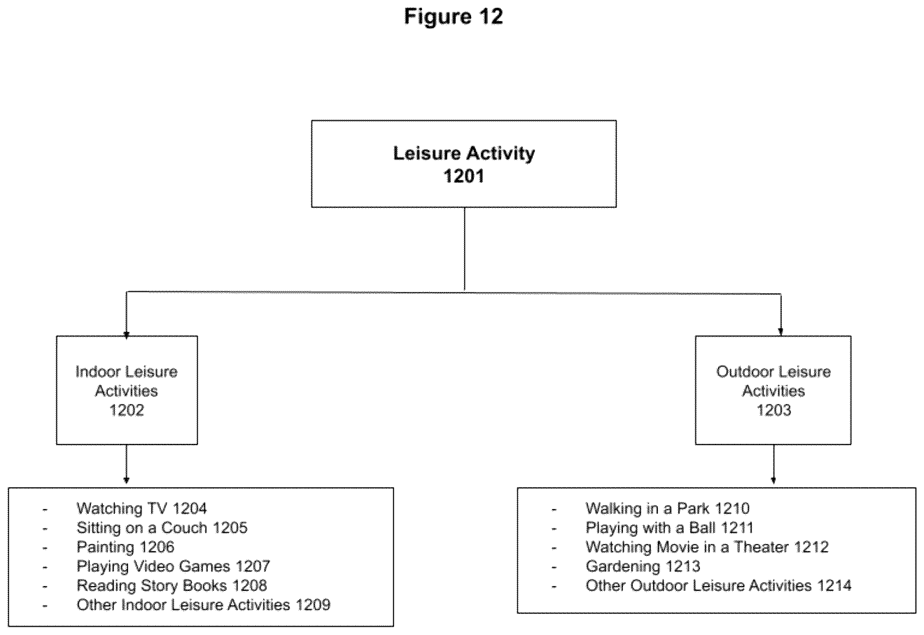

Similarly, there could be examples of various Leisure Activities 705 (See FIG. 12) such as sporting events, going to a park, shopping, eating out and other activities which technology can help achieve freedom and independence for people, but there is a challenge of what to do when the technology may not be working for people with cognitive impairments.

There are Other Activities 706 such as Visit Hospitals 709, Doctors Appointments 710, family visits, and other locations where the need for a system which helps manage when technology is not working is needed.

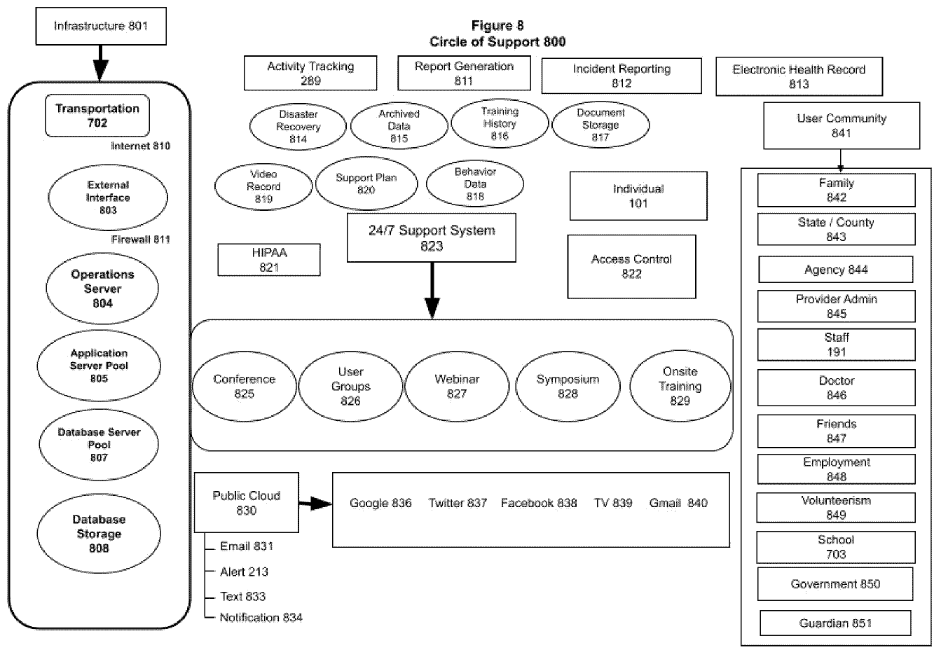

FIG. 8 depicts a circle of support.

FIG. 8 illustrates a Circle of Support 800, which is a way of connecting a person with a disability to their community. It is a collaborative approach to providing assistance and fostering well-being for those individuals. This concept revolves around a group of people who come together to offer emotional, social, and practical support tailored to the Individual's 101 needs.

The circle typically includes User Community 841 under which there are State/County 843, Family 842, Agency 844, Friends 847, Employment 848, Volunteerism 849, School 703, Provider Admin 845, Staff 119, Doctor 846, Government 850, Guardian 851 who may play a role in the person's life. Each member of the circle contributes their unique strengths and resources, working collectively to address challenges and promote the Individual's 101 goals while complying with the standards of HIPAA-type regulations (HIPAA 821).

Infrastructure 801, on the other hand, can refer to the underlying systems and structures necessary for the functioning of a society or organization, such as Transportation 702, Firewall 811, Internet 810, Operations Server 804, External Interface 803, Application Server Pool 805, Database Server Pool 807, Database Storage 808. Communication networks within the Infrastructure 801 allows members of a circle of support to stay in touch and coordinate more effectively.

A 24/7 Support System 823 within a Circle of Support ensures that Individual 101 has access to assistance and care around the clock, which may be continuous. For that reason, Conference 825, User Groups 826, Webinar 827, Symposium 828, Onsite Training 829 are held to support the Individuals 101 with due diligence.

Public Cloud 830 services can significantly enhance a Circle of Support by providing various tools and platforms such as Google 836, Twitter 837, Facebook 838, TV 839, and Gmail 840. These facilitate communication, coordination, and management of support services. Email 831, Alert 213, Text 833, Notifications 834 can be incorporated in these tools and platforms to facilitate the support to Individual 101.

When a support Staff 119 dies within a Circle of Support, it can be a significant and challenging situation for everyone involved. The impact and response will vary depending on the roles and relationships the staff member had with the individual and the rest of the support network. The support circle will need to reassess the Individual's 101 needs and how the gap left by the member of Staff 119 can be filled. This may involve evaluating which aspects of support need immediate attention. In this case, Individual's 101 Document Storage 817, Training History 816, Report Generation 811, Support Plan 820, Video Record 819, Archived Data 815, Activity Tracking 289, Incident Reporting 812, Behavior Data 818, Electronic Health Record 813, Disaster Recovery 814 can assist significantly. These can provide analytics and reporting tools to track health metrics, prove Access Control 822, monitor progress, and assess the effectiveness of care plans. This data can help the circle of support make informed decisions and improve the quality of care by the newly recruited Staff 846, after the death of the previous staff member.

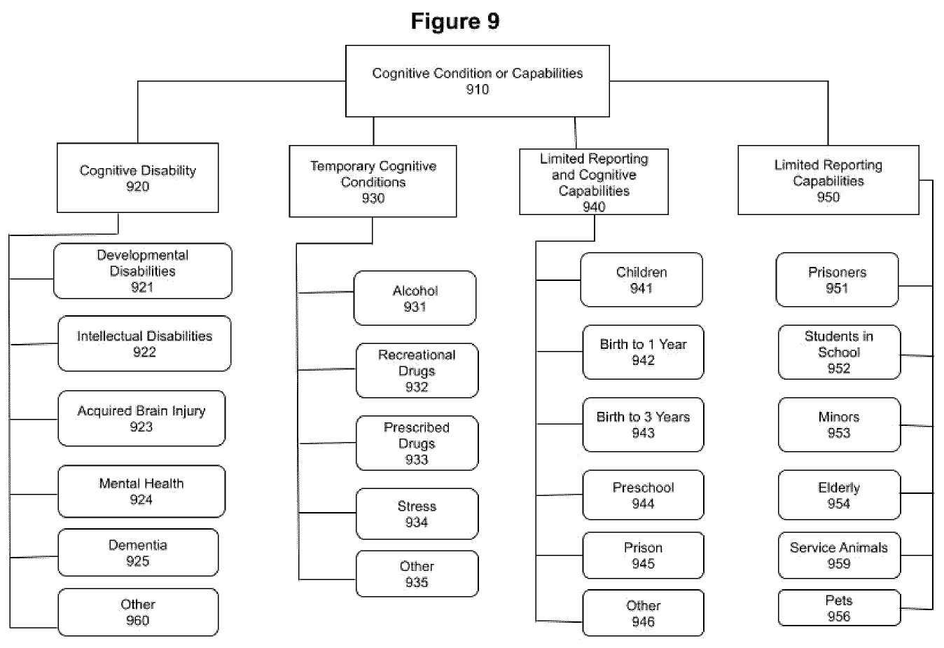

FIG. 9 depicts cognitive conditions.

FIG. 9 depicts cognitive conditions. Cognitive Conditions or Capabilities 910 refer to the state and functioning of an individual's mental processes, which might include memory, attention, reasoning, language comprehension, and decision-making. These capabilities might influence how a person interacts with the world and manages daily tasks.

Individuals with Developmental Disabilities 921 might face challenges from a young age that could affect their learning and social integration. Similarly, those with Intellectual Disabilities 922 might find complex tasks or intricate social interactions particularly challenging. Acquired Brain Injuries 923 could lead to a range of cognitive impairments, which might affect memory, attention, and problem-solving skills. This might alter an individual's ability to function as they did before the injury. Mental Health Issues 924, such as depression or anxiety, could also significantly impact cognitive processes, leading to difficulties in concentration and decision-making.

As individuals age, conditions like Dementia 925 and Alzheimer's disease become more prevalent, leading to a progressive decline in memory and reasoning abilities, which might severely affect daily functioning. Lifestyle factors also play a critical role; excessive Alcohol Consumption 931 and the use of Recreational Drugs 932 could impair judgment and cognitive performance. Even Prescribed Drugs 933 might have side effects that affect cognitive functions, such as causing confusion or slowed reaction times.

Environmental and internal stressors, labeled as Stress 934, could exacerbate cognitive difficulties, which could impact memory and concentration. The cognitive development of Children 941 is crucial and could be influenced by various factors including education and nutrition, requiring early intervention for those at risk of developmental delays. Lastly, the natural aging process seen in the Elderly 954 might lead to a gradual decline in cognitive capabilities, influenced by overall health, mental activity, and social engagement, underscoring the importance of supportive environments to maintain cognitive health.

Cognitive Disabilities: These are impairments in cognitive functioning that could affect an individual's ability to process information, learn new skills, communicate and others. People with cognitive disabilities might experience challenges in academic or work settings and might require specific interventions and support.

Prader-Willi Syndrome: Prader-Willi Syndrome (“PWS”) involves cognitive and consumptive challenges. Individuals with PWS could have varying degrees of intellectual impairment and might exhibit behaviors that complicate learning and social interaction, such as difficulty with change or unstructured situations.

Temporary Cognitive Conditions 930: Conditions such as concussions or transient stress-related impairments could temporarily impact cognitive functions. An individual experiencing these might have difficulties with memory or concentration, which might resolve as the condition improves.

Limited Reporting and Cognitive Capabilities 940: Sometimes, cognitive impairments could lead to limited reporting capabilities 950. This means that an individual might struggle to articulate their thoughts, needs, or experiences effectively, which might impact their ability to communicate accurately in social and clinical settings.

Limited Reporting Capabilities 950: Similar to the above, this refers to reduced ability in expressing or conveying information. This could affect an individual's interaction with healthcare professionals, and might lead to underreporting of symptoms or challenges.

For individuals with cognitive conditions, various scenarios could emerge depending on the severity and nature of their impairments. For example, a person with mild cognitive disability 920 might manage well with minimal support in place, whereas someone with significant impairments may require comprehensive, ongoing assistance to perform daily activities and maintain quality of life.

Navigating these conditions might require tailored approaches that consider the unique capabilities and needs of each individual. Effective support and accommodations could enhance functioning and participation in various life domains, from education and employment to personal relationships and community activities.

FIG. 10 illustrates a school.

FIG. 10 depicts a school 703 environment that utilizes aspects of the invention to improve safety, efficiency, and performance.

The system leverages various components to monitor and enhance the experience of individuals within the school 703 setting. Key locations, denoted as Locations 1002, serve as monitoring zones where activities, interactions, and other dynamics of the school are tracked. These areas might include classrooms, libraries, gymnasiums, or outdoor spaces, providing a holistic view of the environment.

The primary focus of the system is the Student 1003. Each student's activities and progress are categorized under Categories of School 1004, which might include grade levels, special education programs, or extracurricular involvement. Performance at School 1005 captures metrics such as academic achievements, participation, and attendance. These performance indicators are further Tied to Study Documentation 1006, which encapsulates related subcategories, including Performance 1008, Health Issues 1010, Safety 1011, and other key aspects (Other Tie to Study Documentation 1012). For instance, patterns in attendance or declining grades might trigger health-related analyses to determine whether Health Issues 1010 or Safety 1011 concerns need intervention.

Integrated sensors (Sensors to Capture Expected Classes to be Done 1007) play a critical role in monitoring adherence to scheduled activities or identifying deviations from expected routines. For example, the system can detect if a student is missing from a required class or flag irregularities, such as prolonged absence from scheduled academic sessions. Exemplary automated attendance systems and methods that may be integrated with embodiments of this invention are disclosed in the '354 application and are incorporated by reference herein.

Notifications 834 serve as a critical communication framework within the system, encompassing a variety of recipient-specific categories to ensure effective dissemination of information. These include Notify Student 1014, which targets students with reminders, safety alerts, and performance updates, as well as to Guardian 851, which keeps parents or guardians informed about their child's activities, progress, or urgent concerns. Agency 844 notifications address educational authorities or external organizations for operational updates or compliance matters, while Other Individual 1019 focuses on staff and educators, enabling coordination on tasks such as health-related events or emergency drills. For regulatory purposes, Government 850 notifications ensure timely reporting and adherence to compliance requirements.

Additionally, Other Notifications 1018 provides a flexible category for addressing specialized or unforeseen situations, ensuring that all potential scenarios are covered under the notification system. Together, these elements enable a well-rounded, adaptable communication strategy within the school setting depicted in FIG. 10.

FIG. 11 illustrates a job.

FIG. 11 illustrates a job environment integrating aspects of the invention to enhance workplace safety, performance, and communication. The system supports various Locations 1102, representing diverse workplaces, and encompasses different Types of Job 1104 to cater to varied industries and roles. Each job involves an Employer 1103 responsible for overseeing the work environment and ensuring productivity.

To monitor and improve Performance at Job 1105, the invention links tasks to relevant data under Tie to Employment Documentation 1106. This documentation records key elements like Performance 1109 evaluations, Health Issues 1110 impacting job output, Safety 1111 measures, and Other Tie to Employment Documentation 1112 to ensure a comprehensive account of employee interactions with their role.

Innovatively, Sensors to Capture Expected Work to be Done 1107 enable the system to monitor assigned tasks, identifying completion rates, anomalies, or inefficiencies in real-time. This capability aids in supporting employee performance and streamlining work processes.

Notifications 834 act as an integral communication tool, covering categories such as Notify Employer 1113 for updates on employee progress or workplace issues, Agency 844 for compliance alerts or external coordination, and Guardian 851 to inform family members in case of emergencies or critical developments. Additionally, the system supports notifications for Other Individual 1019 employees to guide their actions or provide real-time feedback, Government 850 for compliance reporting, and Other Notifications 1118 to address unique scenarios that require specific communication channels.

Incorporating these components, FIG. 11 highlights how the invention facilitates an efficient, responsive, and supportive job environment, enhancing productivity, safety, and overall work dynamics.

FIG. 12 depicts a leisure activity which might utilize aspects of the invention.

FIG. 12 illustrates a leisure activity 1201. Leisure activity 1201 could include both indoor leisure activities 1202 and outdoor leisure activities 1203. An individual 101 could have different kinds of leisure activities 120. An individual 101 could be watching TV 1204 while sitting on a couch 1205 could do some painting 1206, play video games 1207. They can also read story books 1208 and do other Indoor leisure activities 1209.

Apart from indoor leisure activities 1202, an individual can have outdoor leisure activities 1203 as well. As part of outdoor leisure activities 1203, an individual 101 can be walking in a Park 1210, playing with a ball 1211. Also, they can watch movie in a theater 1212, do gardening 1213 outside of their home. An individual 101 can have other outdoor leisure activities 1214 to pass their quality time.

FIG. 13 depicts sensors which may be used in embodiments of the invention.

FIG. 13 illustrates devices 413 and sensors 121 that may be used in embodiments of the invention. All these devices 413 and sensors 121 can have various types of data to collect 1214. There can be continuous 1301 devices 413 such as sleep mat 1303 and other continuous devices 1350, discrete 1304 devices 413 such as scale 1306 and blood pressure 1307 and other discrete devices 1360. Devices 413 can be wearable 1305 inclusive of watches 1308, clothing 1309, rings 1310, jewelry 1311, footwear and other wearable 1315 devices 413.

Fixed Location 1316 can have some specific refrigerators 1317, lamps 1318, Walls 1319, fixed camera 1320, fixed Sensor 1321, Door 1322; also, there can be other fixed locations devices 1323.

Devices can be in variable location 1324 having drones 1325, movable devices 1326, robots 1327, cameras 1328 and other variable location devices 1329 to monitor the activities of an individual 101.

There are methods of capturing data 1330 through camera 1331, sensor 1332, LiDAR 1333, thermal 1334, RGB 1335, hidden camera 1336, fitness 1337, smart watches 1338, blood glucose devices 1339, facial identification 1340 (as discussed above with reference to FIG. 5G), and apart from these there can be other methods of capturing data 1341 for an individual 101. Each of the different devices and data capture methods may involve multiple Types of Data to Collect 1370.

For example, an individual's 101 activities can be detected through a LIDAR 1333, which stands for Light Detection and Ranging, which is a remote sensing technology that uses laser pulses to measure distances to objects. It operates by emitting a laser beam that travels to the ground and reflects back to the sensor. By measuring the time it takes for the light to return, LIDAR can determine the distance between the sensor and the object, creating a detailed 3D map of the environment. So, if an individual gets injured, a LIDAR can easily detect that it can transmit information to a support staff.

FIG. 14 depicts a bedroom as implemented in embodiments of the invention.

FIG. 14 depicts a Bedroom 311 as implemented in embodiments of the invention, as set up for an Individual 101. A significant venue for a need for information is Bedroom 311. A Bedroom 311 could be a location where one or more people sleep at night and/or during other portions of the day. Bedrooms 311 are used for other activities besides sleeping including meditation, yoga, talking on a telephone, talking to someone in a room, having romantic relations, interacting with one or more people inside the room, having visitors, reading a book, being on a computer or other electronic device, walking to a bathroom, walking to leave the room, playing games in bed, playing games on the floor, sitting the floor and many other activities. Many of these activities are normal and would not require any outside assistance or change in staffing or other changes in the environment.

However, while many of those activities may be without danger, there are many activities which could be dangerous, risky, or potentially dangerous inside a Bedroom 311 venue. The activities could include choking on something in the room, falling from a bed to the ground, falling while walking or standing, leaving the room, exiting a room through a window, playing with an eclectic outlet which could lead to electrocution, playing with a string or wire would could lead to strangulation, incontinence, being near water which could lead to drowning, self-injurious behavior including cutting, or other activities which could be problematic.

In a situation where the Internet 1444, electricity, and other devices are all showing they are properly working according to a Smart Socket Device 1415 the system could monitor activities according to an individual's plan and goals and notify Other Individuals 1471 including Staff 119, Families 842, Friends 847, Guardians 851, or Additional Individuals 1472 take action or other appropriate measures based on sensors and devices in a Bedroom 311. Smart Socket Device 1415 could let the system know that key network components such electricity, Internet 1444, and communication network(s) including telephone service, are active, such as those described in the '316 Patent (and the '074 Publication at Paras. 0037-0041 and FIGS. 1-2 for basic function, and 0054-0065 and FIGS. 11-16 for specifics of an exemplary device).

Devices could be monitoring sleep through a bed monitor, an infrared sensor, a Camera 1410, a ring, a watch, or many other sleep monitoring devices. Methods of monitoring could include some form of sleep mat under the mattress, over the mattress, built into the mattress, on top of the sheet, built into a sheet or blanket or other forms of monitoring. These devices could need some form of electric power, electricity, or batteries to monitor the condition and then report on the condition through Wi-Fi 1443, Bluetooth 1442, or other method to a sensor which could determine action to be taken.

Other forms of sleeping could include a Crib 1429 for an infant or toddler, a Futon 1453, a Box-Spring 1437, a Mattress 1435, a waterbed, a sofa, a couch, an air bed or air mattress, a floating bed, a hammock, or other type of beds. Different types of beds or sleeping arrangements could have different needs for electricity and connectivity to convey the information needed.

A Bedroom 311 could have a Bed 1431 which could have one or more of a Box-spring 1427, Mattress 1435, sheets, blanket, or other components.

In a Bedroom 311 for a person with disabilities, there are different kinds of sensors to monitor the vitals of the Individual 101. In FIG. 14 there are sensors like LiDAR 1409, Radar 1413, Smart Socket Device 1415, Sensor Device 1414, Bed Sensor Device 1493, Temperature & Humidity 1411, Sleep Mat 1490. Also, this Bedroom 311 has devices like Wi-Fi 1443, Smart Watch 1489, Power Outlet 1485, TV 1424, Clock 1491. For General furniture there is a Bed 1431, Floor Mat 1422, Light 1421, Chair 1488, Door 1477, Sleepers 1487.

FIGS. 14A-14F illustrate how embodiments of this invention may be used to assist an individual in a Bedroom 311.

FIG. 14A is another view of the Bedroom 311. Individual 101 is sleeping in Bed 1431 and it is 7:30 am in the morning.

Then at 7:31 am the Individual 101 woke up from sleeping (FIG. 14B), but when the Individual wanted to get up from Bed 1431, the Individual tripped and fell on the floor on the Floor Mat 1422 (FIG. 14C). When the Individual fell on the floor, the Individual's Smart Watch 1489 detected the fall and sent a signal to the Staff 1476 who was in the other corner of the house. The Individual got an alert on Smartwatch 1489 (FIG. 14D). Then the Staff 1476 came to the Individual's Bedroom 311 to help Individual at 7.34 am (FIG. 14E). Because of the fall detection and the signal that was sent from Individual's Smart Watch 1489, the Staff could take fast action to help the Individual.

The discussion of Bedroom 311 as described above may also assist with an individual's sleeping habits. The concept of sleeping means different things to different people. Technology has evolved, as has the understanding both the importance of sleep and what occurs while people are sleeping. There are products on the market and people developed which help address sleep issues. Products such as Fitbit, Google Watch, Apple Watch, Aura Ring, Withings Sleep Mat, and many other products provide real time, near real time, delayed, on-demand, push and pull notifications about various sleep related measures. Common data points can include sleep scores, time in Rapid Eye Movement (REM) sleep, heart rate variability, blood oxygen, heart rate, body temperature, snoring, time restless, amount of time in bed, date/time out of bed, date/time in bed, movement, bed exits, breath rate, time in non-rapid eye movement (NREM). Products are measuring them by many methods, including brain waves and polysomnography tests.

There are lists of things people can do to improve their sleep. Casper. com, a mattress company, on their website has a list of things people can do to improve their sleep including working out daily, eating more fiber (it assumes people don't eat enough fiber, some people might eat enough so the word “more” fiber probably has an upper limit), performing cyclic meditation or yoga, avoiding caffeine, avoiding alcohol, having a room at the right temperature, having a comfortable mattress (which they sell), listening to white noise or pink noise, listening to binaural beats which means each ear hears different music, and using an eye mask to block light. Other websites, sleep experts and others have additional suggestions for better sleep.

Some or all of these activities or actions could be appropriate for different people. In general, there is an understanding that improved sleep leads to better health.

Embodiments of this application combined with existing external communal devices can help monitor many of these suggestions. Some suggestions are related to activities actually taking place in a Bedroom 311 such as having a comfortable mattress, while other suggestions such as monitoring food would be monitoring activities during the remainder of the day. With a complete integrated documentation system, these activities could be tracked. When there is a change in planned behavior support staff, families, or caregivers could be notified. However, when there is a change in expected behavior due to problems with power or connectivity, it could affect someone's sleep without notice to caregivers.

There are increasingly products, devices and apps which help monitor many areas which can lead to more sleep. For example, Megelin.com has a “smart anti-snore pillow, there are six airbags and a snore detection sensor. Each airbag has a Head-position sensor underneath. When the snore sensor hears snoring, it tells the controller box. The controller then tells the Head-position sensors to inflate the airbags, which turns your head to the side. This helps you stop snoring by making you sleep on your side.”

There are other products which are positioned to help protect against sleep apnea and other snoring related issues. CPAP devices or Continuous Positive Airway Pressure devices can be a treatment option. These can require a prescription from a doctor. The CPAP machine will need to be set up in a proper manner. Depending on models, the CPAP machine could shut off if electricity ceases to function. There could be backup power options including a battery pack.

Embodiments of this system will help monitor many of these actions to help ensure sleep in compliance with an individual's goals and plans.

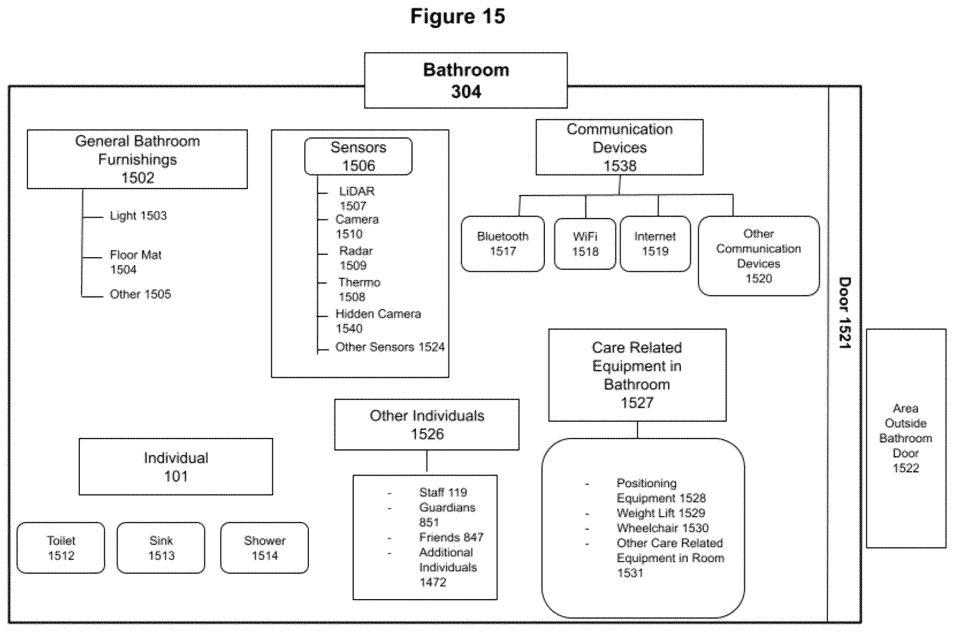

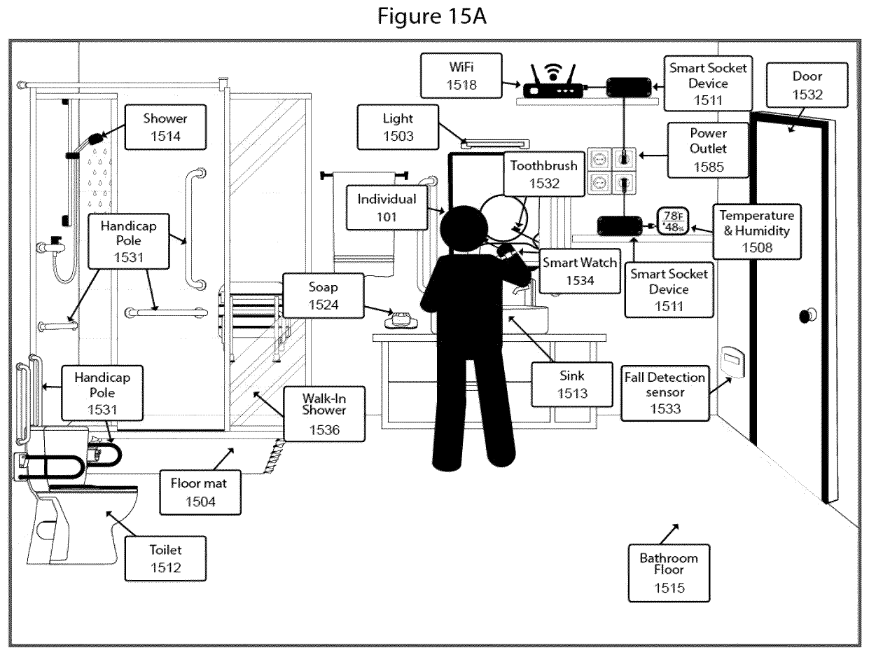

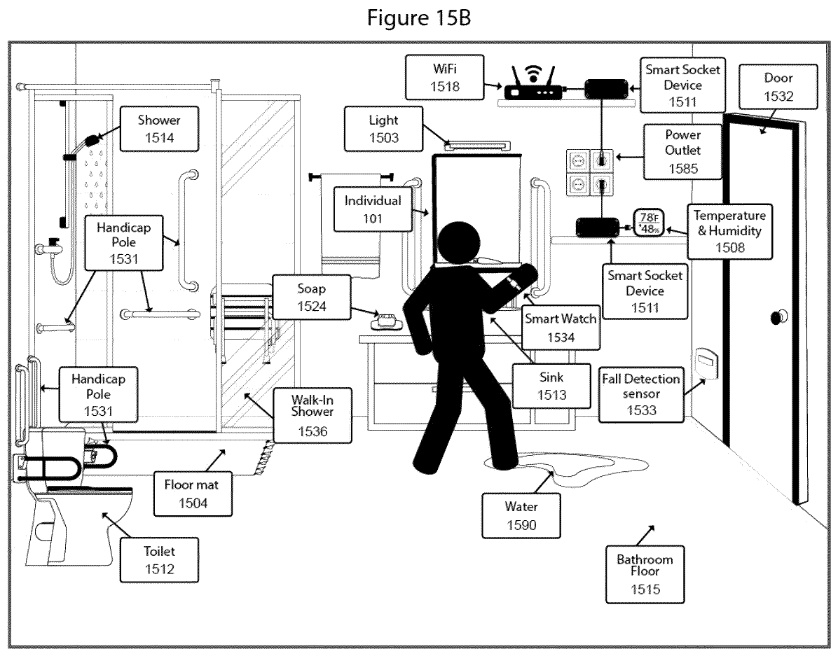

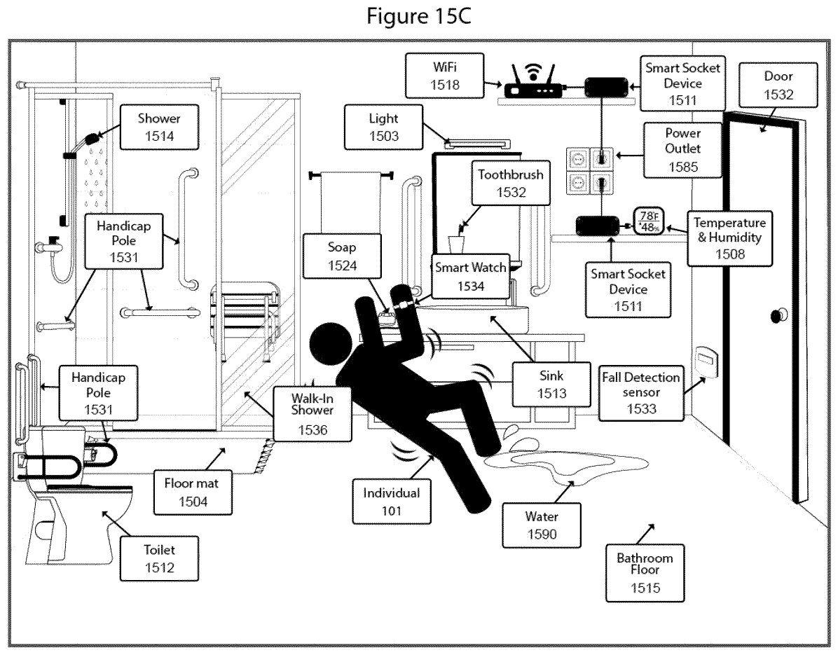

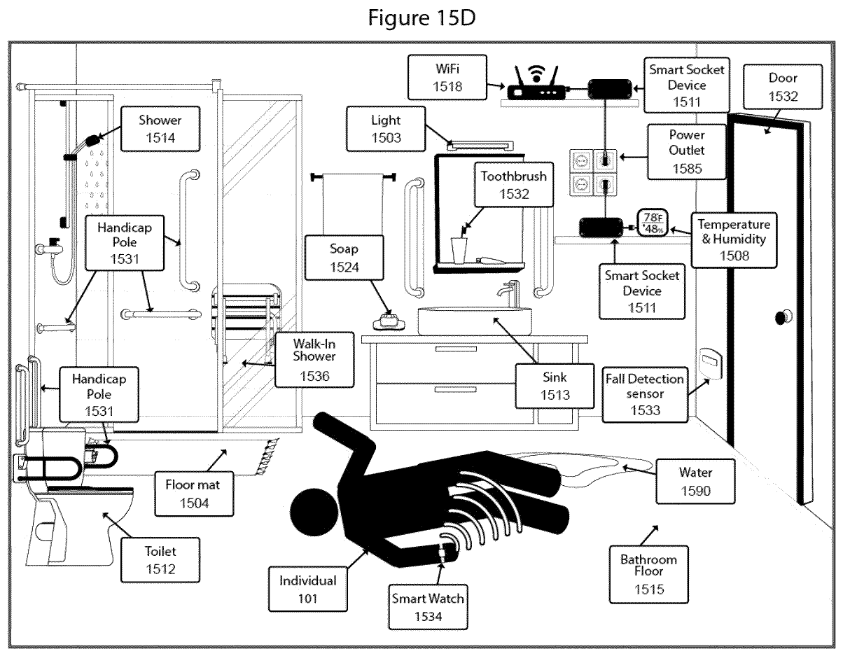

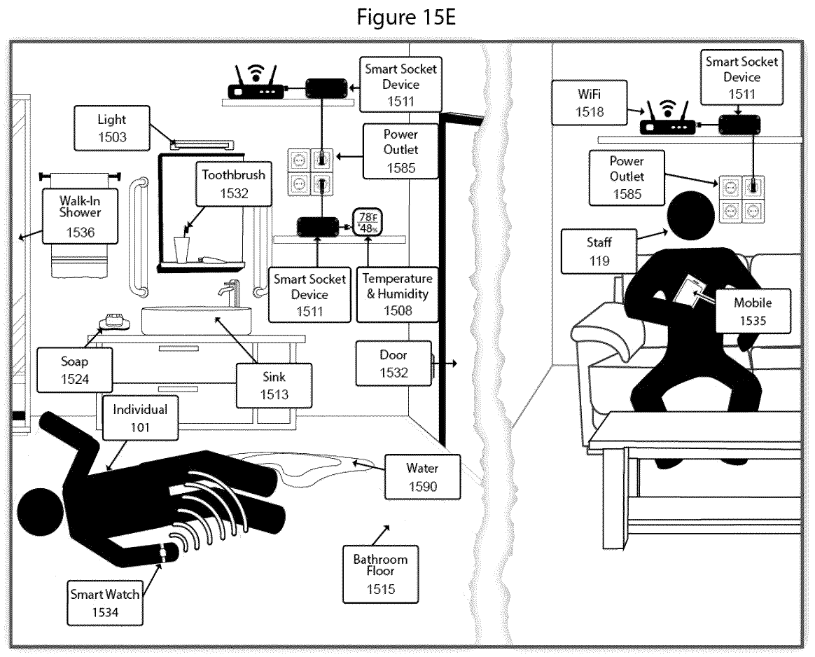

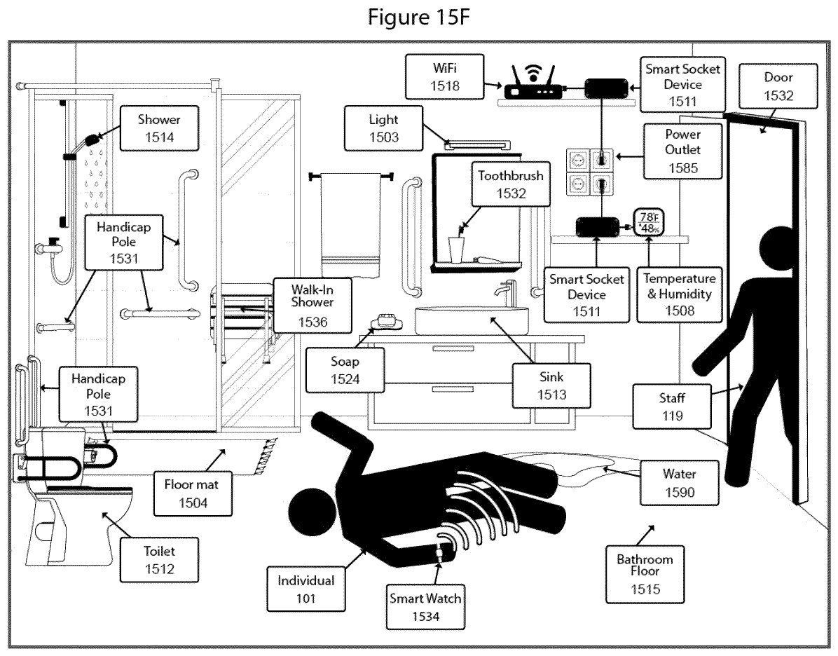

FIG. 15 depicts a Bathroom 304 as implemented in embodiments of the invention, as may be used by Individual 101.

While a Bathroom 304 has many similar documentation issues as a bedroom 311, there are some significant differences. First of all, people preferably should be awake inside Bathroom 304. For example, there could be an expectation that if they are on the Floor 1515 that something could be problematic. A Bathroom 304 typically has smaller dimensions than a Bedroom 311. There could be water in a Bathroom 304—either in a Sink 1513, a toilet bowl, or a bathtub so there is a higher possibility of drawing in a Bathroom 304 than one would expect in a Bedroom 311.

Bathrooms 304 can be inside a single individual person's bedroom, inside a multiple-person bedroom or in a hallway or another room in a home. If getting to a bathroom requires going out a bedroom door through a hallway or other room then a hallway sensor device could notify a staff or system that someone is up and using a bathroom, this could be useful at night when they are supposed to be asleep and a staff might be in another room of the house.

A bathroom could have a Sink 1513. A Sink 1513 could have hot and cold water. There could be Power Outlets 1585 in Bathroom 304 and also near a Sink 1513. The combination of water and electricity could create the risk of electrocution. A bathroom could have a Bathtub or other large vessel which can accumulate water at a level high enough to cause drowning. A bathtub and water on the ground could create a slip or fall. A slip or fall could cause a head injury, a body injury, bleeding, or other situation where someone would need assistance.

There are other potential hazards in Bathroom 304. There could be mats on the floor which someone could slip on, there could be a dry bar or wet bar of Soap 1524 on the Floor 1515, there could be razors or scissors which could be injurious, there could be medications in a cabinet, there could be cabinets filled with different cleaning ingredients, and other type of materials in a Bathroom 304.

While some individuals might be permitted to go to a Bathroom 304 independently, there could be a situation where individuals need to have one or more Other Individuals 1526 a member of Staff 119, Family 842 member, Guardians 851, Friends 847, or Additional Individuals 1472 aware of or assisting when they use Bathroom 304. Someone might be expected to leave a door open or alert someone when they enter and leave Bathroom 304.

There are privacy issues related to the use of Bathroom 304. People could be in various states of undress or not wearing clothing. There may be a concern about photographs or video from within Bathroom 304.

While the expectation could be that hazardous materials are not left out, especially in a group supported home environment it is possible that someone left something out. Or something that is dangerous to one Individual 101 would not be a problem for Other Individuals 1526. However, Staff 119 or Families 842 could be relying on cameras, Sensors 1506, or other devices to help monitor events inside a Bathroom 304. These devices can analyze what is going on inside a room based on a combination of caseloads, individual plans, cameras, Sensors 1506, computing analysis and other information and as needed alert appropriate Staff 119 or take actions. In conditions where the capability to communicate actions to be taken is down, there could be a need for staff outside a room to be alerted that they have lost the ability to track what has happened inside the room.

FIGS. 15A-15F illustrate how embodiments of this invention may be used to assist an individual in a Bathroom 304.

The Bathroom 304 depicted in FIG. 15A is designed so that it can ensure safety and accessibility for individuals with disabilities. Bathroom 1500 can feature a range of equipment and components including Handicap Poles (1531), Shower (1514) and Bathtub (1536), Floor Mat (1504), Toilet (1512), Sink (1513), Fall Detection Sensor (1533), Temperature and Humidity Device (1508), Lighting (1503), Smart Watch (1489) and Wi-Fi (1518), Smart Socket Devices (1511), Power Outlet (1585), Soap (1524), and others.

Despite the comprehensive bathroom setup designed to cater to both daily needs and emergency scenarios for individuals with disabilities, accidents can still occur. For example, Individual 101 can go to the bathroom to brush their teeth at night, and while doing so, the Individual 101 could accidentally spill Water 1590 on Bathroom Floor 1515 (FIG. 15B). This can lead to an accident, as the Individual 101 might slip on the wet floor and fall (FIG. 15C). Since the Individual 101 can be equipped with a Smart Watch 1489 on their wrist and there can be a Fall Detection Sensor (1533) installed in the bathroom, these devices can promptly alert caregivers or medical personnel to ensure immediate assistance is provided (FIG. 15D). The Support Staff 119, who might be anywhere in the house, can receive alerts on their Mobile Device 1535 through the aid of Smart Socket Device (1511) or Wi-Fi 1518 (FIG. 15E), and quickly come into the bathroom to ensure that immediate assistance is provided (FIG. 15F).

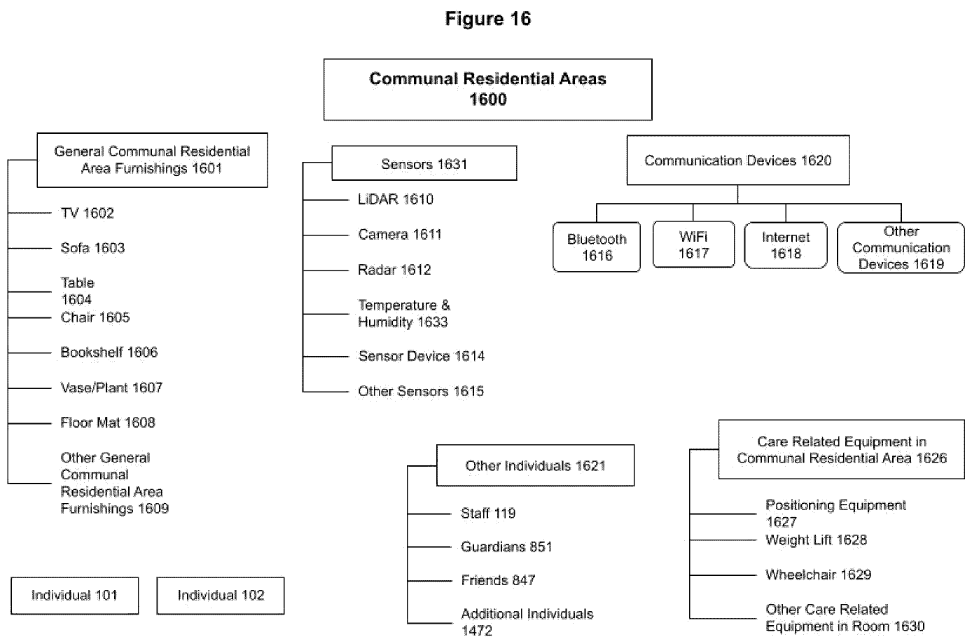

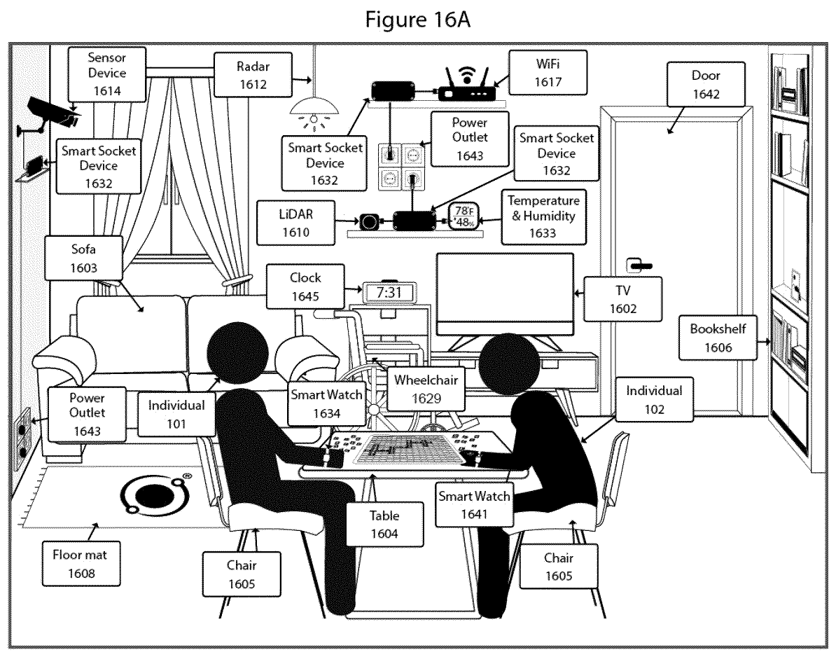

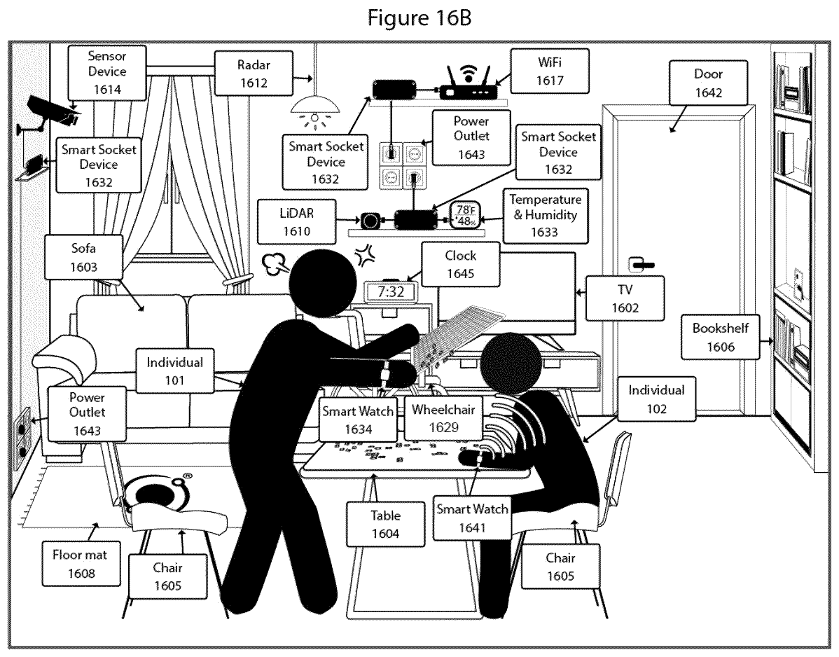

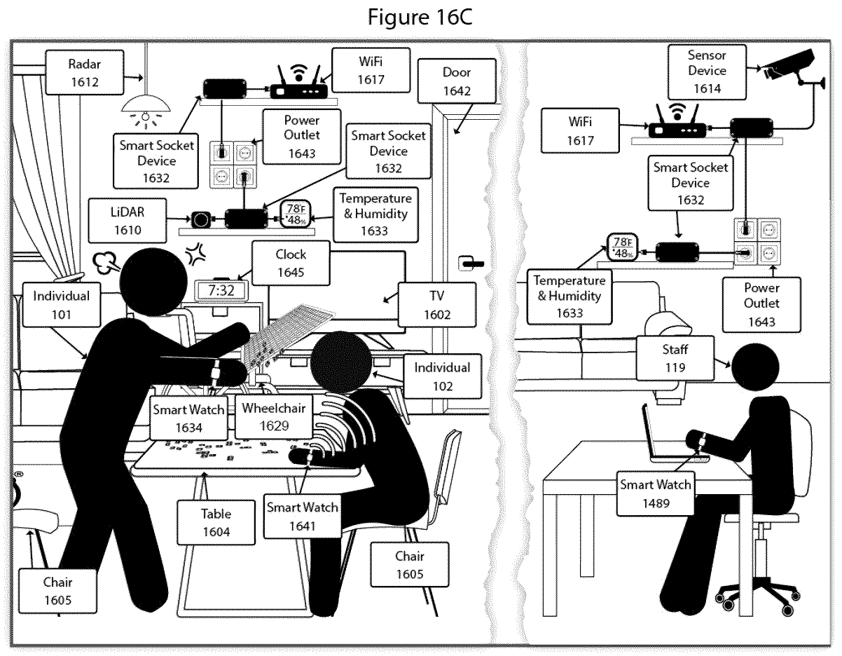

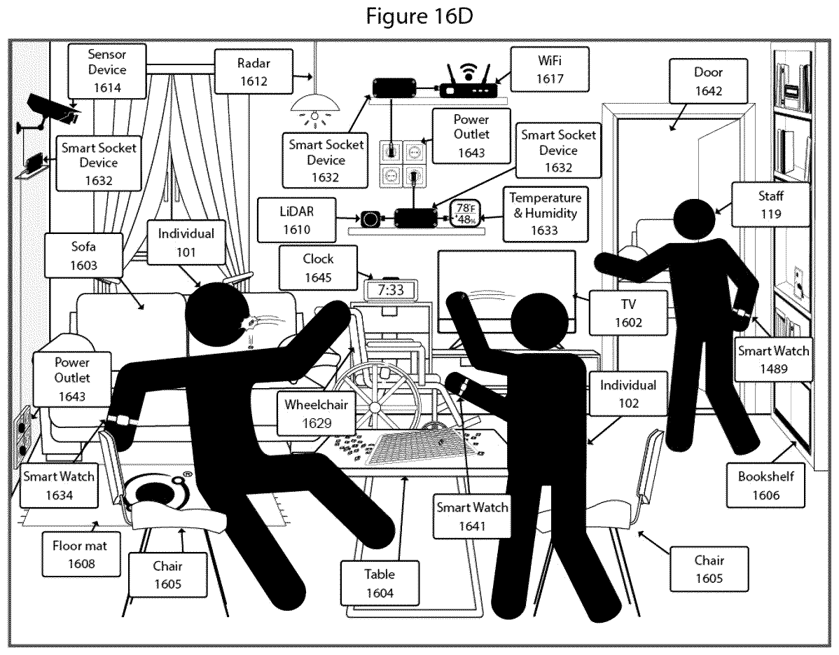

FIG. 16 depicts a Communal Residential Area 1600 in a residence which may be occupied by members of one or more families including where there is organized or paid staff providing services and support to residents.

There are many different names which Communal Residential Areas 1600 may be called, such as family rooms (i.e. family room 307), living rooms, dens, common rooms, shared living spaces, group housing units, joint living quarters and other names. A Communal Residential Area 1600 is a multi-purpose space designed for activities including common social activities, meetings, training, and individual activities as well as other activities which might occur in the space. It can either be open-ended or enclosed, with the open-ended area being linked to the bedrooms or family rooms area where Staff 191 or Guardian 851 can see what the Individuals 101 are doing. Communal Residential Areas 1600 in a residence generally includes coffee tables, side tables, sofas, armchairs, ottoman, bookshelves, cabinets and so on. It may feature decor elements including but not limited to area rugs, pillows, blankets, indoor plants, wall art, chandeliers, and lamps. Entertainment options like a television, speakers, gaming consoles, and board games may also be part of the setup. People may go to Communal Residential Areas 1600 by themselves or with a group of other people to have conversations, watch TV, read books, play games and so on.

While most of these activities are generally safe, any of them could involve risks to one or more people depending on the situation. An individual could have a medical condition (physical or behavioral) which could be triggered or occur not related to the actual activity being engaged in. For example, someone could trip or fall independent of any problems with the physical environment they are in. For individuals 101 or groups of people, there are potential hazards that can arise in Communal Residential Areas 1600. The elements present may incur physical risks such as uneven floors or loose rugs, sharp edged or unsecured furniture; For example, Individuals might trip on uneven floors and fall abruptly. Sensory overload from excessive noise or bright lights can be distressing for individuals with sensory sensitivities. Individuals may become protective over shared items like TV remote, games, pillows. There could be fire safety concerns, including improper use of devices leading to electrical hazards, unclear exits or inaccessible fire alarms and extinguishers, which could pose additional challenges. Lack of privacy or social isolation could negatively impact Individuals' emotional well-being. For example, an Individual might want to enjoy time alone reading a book and a crowded space could lead to feelings of distress. These hazards could compromise the safety and overall quality of life in the home if not carefully addressed.

In order to prevent these from happening, installation of sensors or monitoring devices might be required in the Communal Residential Area 1600 and Individual 101 could also wear monitoring devices themselves for their safety. These devices are programmed to monitor and record individuals based on pre-set rules. Upon detecting an incident, these devices could quickly alert one or more Other Individuals 1621 including the support Staff 119, Guardians 851, Friends 847, Additional Individuals 1472, or someone responsible for overseeing Individual 101. Sensors 1631 such as LiDARs 1610, Cameras 1611, body cam, facial ID, Sensor Devices 1614, Temperature & Humidity 1633, Radar 1612, infrared cameras, pet animals, robots, and other monitoring devices could be installed in the Communal Residential Area 1600. These devices could monitor the situation and send reports to the staff through smartwatches, rings, mobile videos, and others. For example, if two Individuals start fighting while playing a game such as Scrabble, the support staff 119 monitoring the individual through a Camera 1611 could be notified of the outburst. To function effectively, these devices require technological connectivity and depend on electric power, batteries, or other energy sources to transmit data through Wi-Fi 1617, Bluetooth 1616, or other Communication Devices 1619 to Sensors 1631.

In case of an electricity or Internet 1618 blackout or other situations where it is impossible for Staff 119 to use technology to monitor the Individual, alternative safety protocols preferably could or should be activated. This includes manual supervision at regular intervals, and the use of battery-powered emergency systems to ensure continuous monitoring and response. For example, battery-operated motion sensors could trigger an alarm if unexpected movement is detected, alerting nearby personnel even when digital systems are down.

FIGS. 16A-16D illustrate how embodiments of this invention may be used to assist individuals in a Communal Residential Area 1600.

In FIG. 16A, the Individual 101 and Individual 102 are placing the Scrabble Letter Tiles at the same time while playing on a Table 1604. They are wearing smartwatches. Communal Residential Area 1600 has General Communal Residential Area Furnishings 1601 with Sofas 1603, Pillows 1635, Table 1604, Chairs 1605, Floor mat 1608, Plant 1607, Table Lamp 1636, Light 1637, TV 1602, PlayStation 1638, Gaming Consoles 1639, Bookshelf 1606. Multiple Sensors 1631 are set around Individual 101 and Individual 102 to track down the Individuals' activities such as LIDAR 1610, Camera 1611, Sensor Device 1614, Radar 1612. Smart Socket Device 1632, Power Outlet 1643, Temperature & Humidity 1633 are also located in this scenario.

FIG. 16B shows that Individual 101 and Individual 102 are getting angry at each other and the situation is escalating when Individual 101 picks the game board and slams it on the head of Individual 102.

In FIG. 16C, Support Staff 119 can be seen in another room, which may be near Communal Residential Area 1600. Staff 119 has Wi-Fi 1617, Smart Socket Device 1632, Temperature & Humidity 1633, and LiDAR 1610 in that room. While Individual 102 has gotten hurt, the Smartwatch 1641 he is wearing is immediately sending a signal to Staff 119 standing in another room.

FIG. 16D shows that after getting the signal, Support Staff 119 immediately arrives to help Individual 102. Individual 102 becomes furious by this sudden incident and starts throwing the rest of the game pieces at Individual 101.

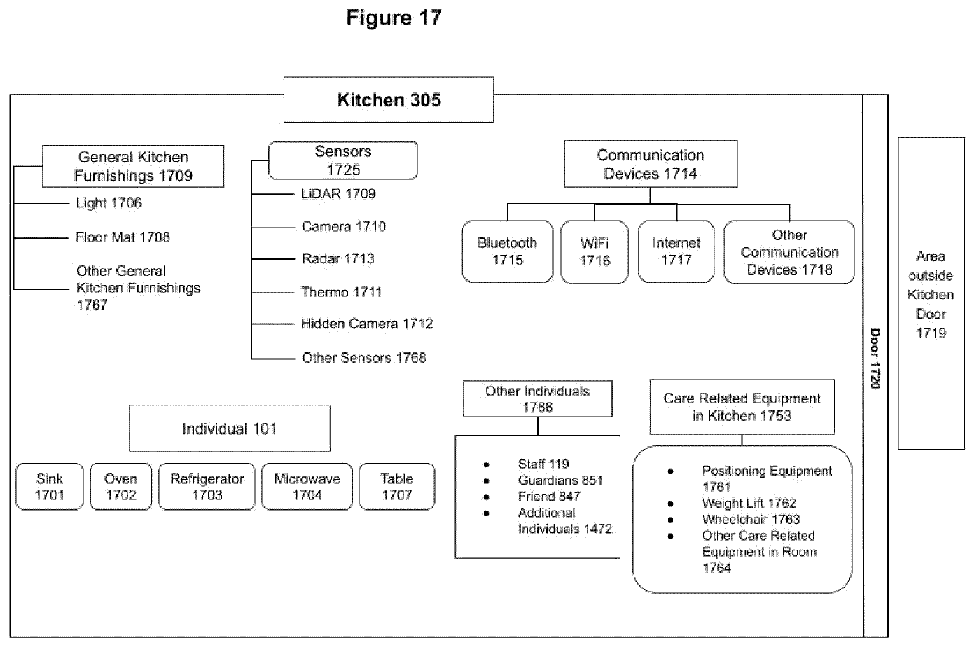

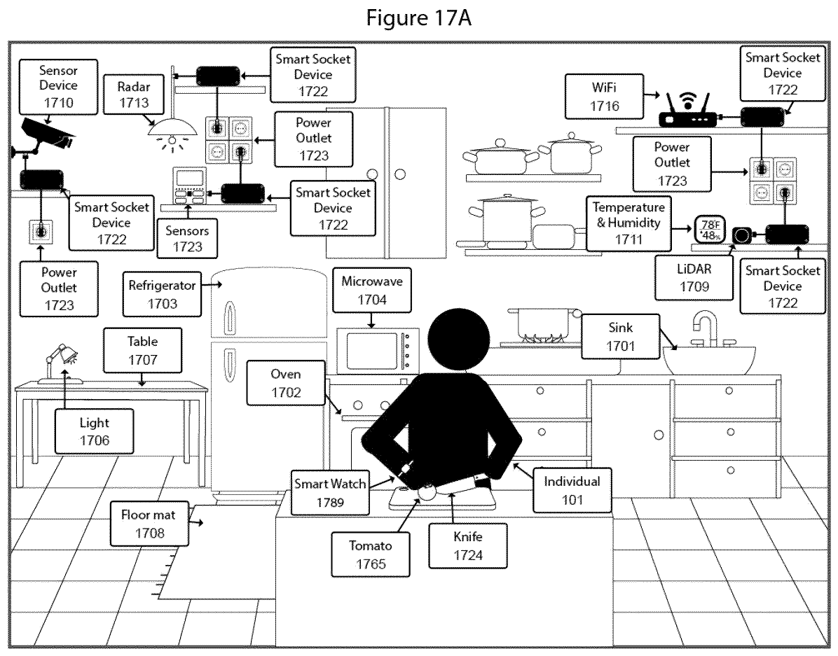

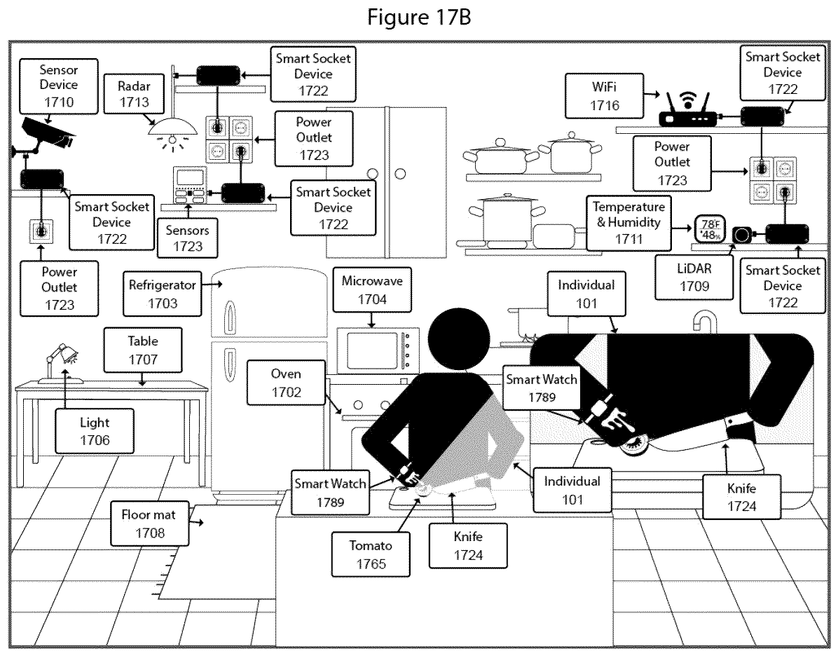

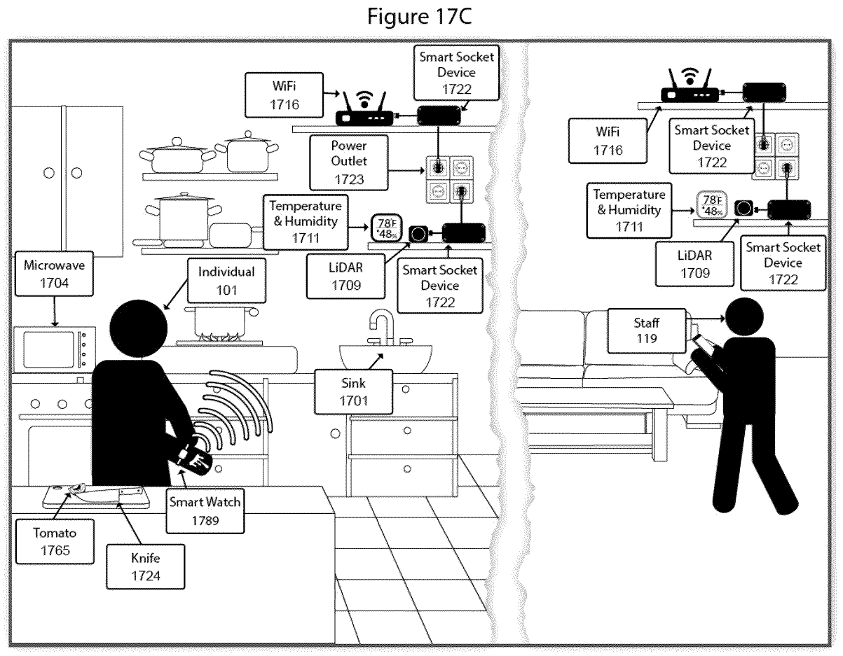

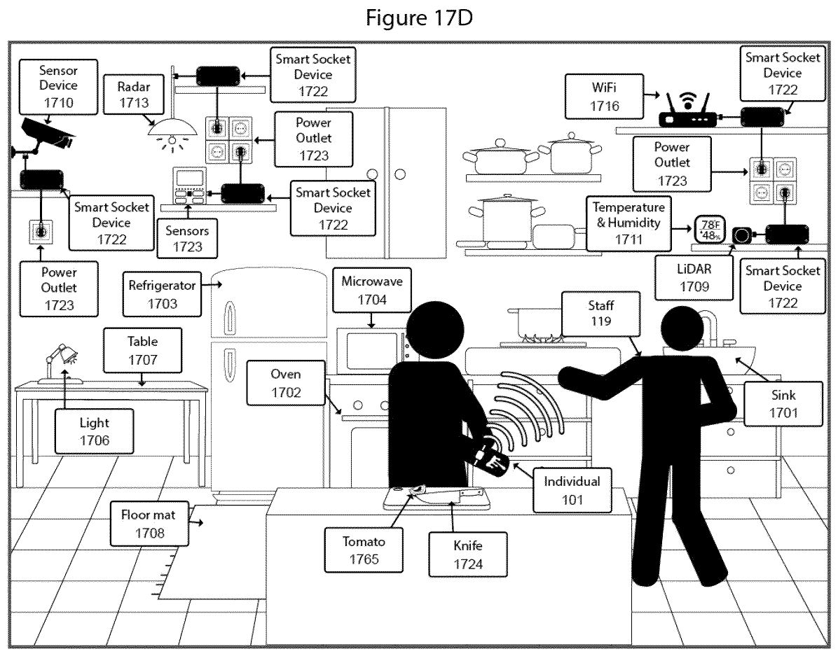

FIG. 17 depicts a Kitchen 305 as implemented in embodiments of the invention, as may be used by Individual 101.

There are multiple uses for Kitchens 305. Kitchen 305 is a place that people use to prepare and eat food, a place for gatherings and talking to other people, it can also be used as an additional area where people can work. Kitchens 305 can either be open-ended or enclosed, with the open-ended area being linked to the living or dining area where Staff 119 or Guardian 851 can see what the Individuals 101 are doing. Kitchens 305 in a household include a Refrigerator 1703, Gas Ovens 1702, Microwaves 1704, stoves, pots and pans, Knives 1724, utensils such as spatulas, whisks, or tongs, a dishwasher, countertops, drawers, a Kitchen Table 1705, Sink 1701, soap, sponges, chemical substances, glass utensils, Rugs or Floor Mats 1708, Light Fixtures 1706, and others. Kitchens 305 are often used for prepping food, cooking, baking, and eating. People can go to Kitchens 305 by themselves or with a group of other people to prepare and eat food or just talk to each other; an individual could go in there to eat food or have a glass of water.

For individuals 101 or groups of people, Kitchens 305 can be hazardous as there are many components present that can cause them harm, such as Knives 1724, forks, stoves, chemical substances, wet sponges, Gas Ovens 1702, electrical circuits, and others. For example, Individuals could accidentally drop glass utensils and cut themselves, might start a fire or get burns from the stovetop, or cut themselves while cutting fruits; there could be a wet sponge or water on the floor for them to slip and fall. Kitchens 305 can also have various chemical substances for cleaning, they could be mishandled by Individual 101, or others including support staff and family members. Fires and electrical hazards can also pose significant risks while someone is in Kitchen 305. For instance, Individual 101 could accidentally spill boiling water on themselves or inadvertently touch an electrical appliance with wet hands, both of which can lead to dangerous situations. Kitchens 305 can also have a Table 1705 where a group of people can eat their food, and chat with each other, or use Table 1705 as their workstation, for example someone can bring in their laptop and work on Kitchen Table 1705.

On account of preventing these accidents, installation of sensors or monitoring devices might be required in the Kitchen 305 and Individual 101 can also wear monitoring devices themselves for their safety. These devices are programmed to monitor and record individuals based on pre-set rules. Upon detecting an incident, these devices will quickly alert one or more Other Individuals 1766 including the Support Staff 119, Guardians 851, Friends 847, Additional Individuals 1472, or anyone responsible for overseeing Individual 101. Sensor Devices 1723 such as LiDARs 1709, Cameras 1710, body cam, facial ID, Hidden Cameras 1712, Thermal Sensors 1711, Radar 1713, infrared cameras, service animals, robots, and other monitoring devices could be installed in the Kitchen 305. These devices would monitor the situation and send reports to the staff through smart watches, rings, mobile videos, barks, and others. For example, if someone accidentally cuts their hand while preparing food, the support staff monitoring the individual through a Camera 1710 will immediately be notified of the injury, To function effectively, most of these devices require technological connectivity and depend on electric power, batteries, or other energy sources to transmit data through Wi-Fi 1716, Bluetooth 1715, or other Communication Methods 1714 to a Sensor System 1723.

In case of an electricity or Internet 1717 blackout or any other situations where it is impossible for the staff to use technology to monitor the individual, alternative safety protocols preferably could or should be activated. This includes manual supervision at regular intervals, and the use of battery-powered emergency systems to ensure continuous monitoring and response. For example, battery-operated motion sensors can trigger an alarm if unexpected movement is detected, alerting nearby personnel even when digital systems are down.

FIGS. 17A-17D illustrate how embodiments of this invention may be used to assist an individual in a Kitchen 305.

In FIG. 17A, the Individual 101 is cutting a Tomato 1765 with a Knife 1724. He was wearing a Smartwatch 1789. Kitchen 305 has General Kitchen Furnishings 1709 with Light 1706, Table 1705, Floor mat 1708, Light 1706, Floor mat 1708. Many Sensor devices 1723 are set around Individual 101 to track down the Individual's activities such as LiDAR 1709, Camera 1710, Thermo 1711, Hidden Camera 1712, Radar 1713. Smart Socket Device 1722, Power Outlet 1723, Temperature & Humidity 1711 are also located in this scenario.

FIG. 17B shows that while cutting the Tomato 1765 with a Knife 1724, the Individual 101 suddenly lost his balance and cut his hand.

In FIG. 17C, Support Staff A 119 can be seen in another room. The staff has Wi-Fi 1716, Smart Socket Device 1722, Temperature & Humidity 1711, and LiDAR 1709 in that room. While Individual 101 cut his hand, the Smart Watch 1789 he was wearing immediately sent a signal to the Staff standing in another room.

FIG. 17D shows that after getting the signal, Support Staff 119 immediately went to help Individual 101. Individual 10 was shocked by this sudden incident and could not move from his position.

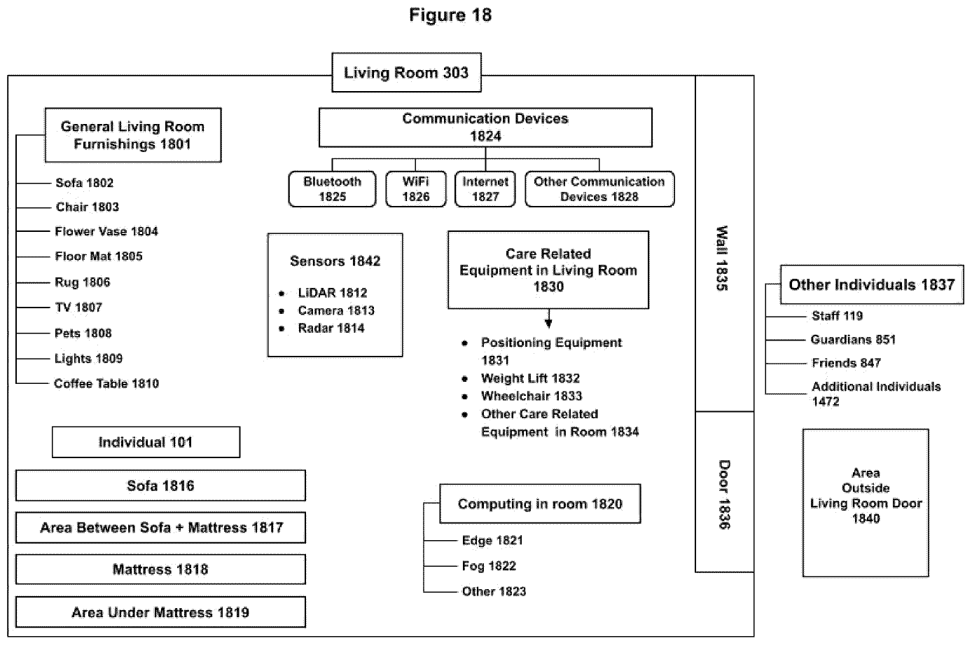

FIG. 18 depicts a living room 303 as implemented in embodiments of the invention, as may be used by Individual 101.

The Living Room 303, den, common area, drawing room, or parlor is a space in a household for relaxation and gatherings for people including family visitors and staff members. The area is furnished with Sofas 1802, couches, TV 1807, sound systems, decorative plants, throw pillows, cushions, Rugs 1806, Ottomans, Light Fixtures 1809, Coffee Table 1810, candles, and other decorative elements 1801. Common hazards in Living Rooms 303 include electrical malfunctions, such as a TV 1807 that shorts out due to faulty wiring, or individuals might trip over loose Carpets 1806 or any loose and exposed wires. For example, a guest could stumble over an unsecured Rug 1806 or a phone charging cable stretched across the walkway. Injuries may occur from sharp-edged furniture like the corner of a metal Coffee Table 1810. There is also a risk of fires from faulty electronics or candles; for instance, a candle left burning unattended might ignite nearby curtains, or an overheating Laptop 1820 could spark a fire.

Living Room 303 preferably could or should also integrate safety and monitoring mechanisms, particularly for households with elderly residents 1837, children, or Individuals 1815 with developmental, intellectual, or cognitive disabilities. To address these safety concerns, many households may employ installations of monitoring sensors or devices such as cameras 1813 or hidden cameras, wearable devices like body cams and smart watches, LiDAR, RGB sensors, thermal sensors, facial identification technology, or other devices. These devices can monitor the situation and alert Other Individuals 1837, including the support Staff 119 or Guardian 851 caring for the individual 101, as well as Friends 847 or Additional Individuals 1472, if an incident takes place. For instance, a thermal sensor might detect an unusually high temperature near the fireplace, signaling a potential fire hazard, and immediately alert the support staff through a connected smartphone app.

Although the above-mentioned monitoring devices may require an internet connection, Bluetooth 1825, electricity, or other technological support to function, there are contingencies for power or connectivity outages. In scenarios where electrical power or internet connectivity is disrupted, alternative safety protocols preferably could or should be activated. These include maintaining clear pathways to exits and ensuring easy access to manually operated emergency equipment like fire extinguishers, and using battery-operated backup systems to keep essential monitoring devices running.

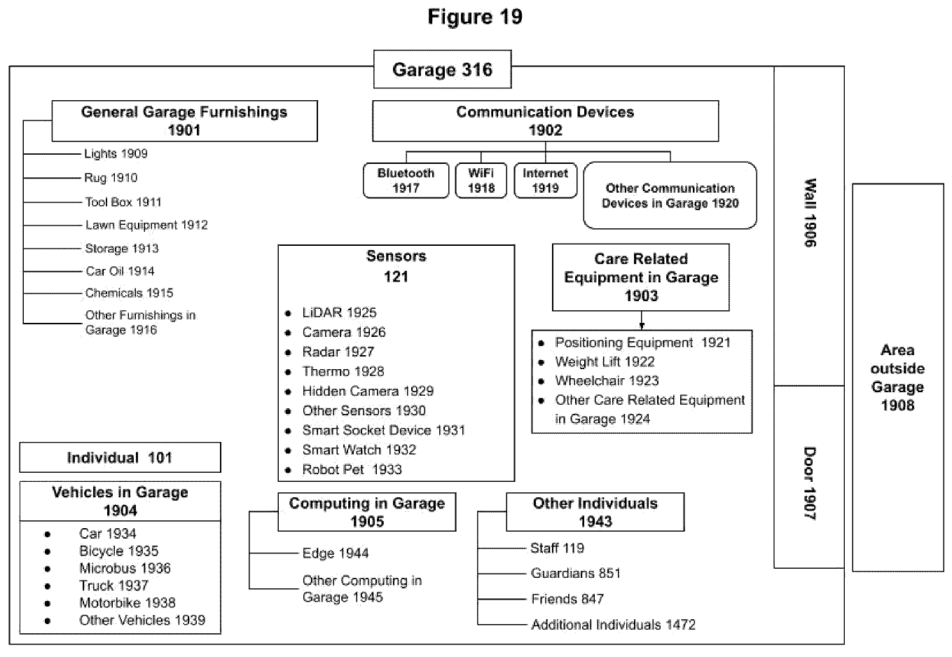

FIG. 19 depicts a Garage 316 as implemented in embodiments of the invention, as may be used by Individual 101.

Garages 316 are spaces that are used for parking Cars 1934 and various kinds of storages and activities. Garage 316 includes elements like an automatic door, workbenches, Tools box 1911, Other Vehicles 1939, Car 1934, Bicycle 1935, Lawn Equipment 1912, Chemicals 1915 for vehicle maintenance, Storage 1913 Units and Other Furnishings in Garage 1916.

Garages 316 can be attached to the house or detached, meaning they can be accessible from inside in some houses and from outside for some.

For Individuals 101, garages 316 can present numerous hazards. The array of tools and machinery, combined with Chemicals 1915 such as antifreeze, paint, and solvents, poses significant risks. People can accidentally injure themselves with tools, suffer chemical burns, or experience slips and falls on oily surfaces. Sometimes accidents like carbon monoxide poisoning if cars are left running in an enclosed space or injuries from moving vehicles can also occur if not careful enough, or maybe individuals can get burns from touching exposed wiring or improperly managed electronic tools.

To mitigate these risks, safety protocols may involve the installation of Sensors 121 or Monitoring Devices 115 such as motion sensors, carbon monoxide detectors, Video Cameras 1926, Thermal Sensors, and Other Sensors 1930 to oversee activities and alert Other Individuals 1943, including Support Staff 119 or Guardians 851, as well as Friends 847 or Additional Individuals 1472, when an incident occurs. These devices typically may require electricity, internet connectivity, or other technological power to function but may also be backed up by battery systems to maintain functionality during power outages.

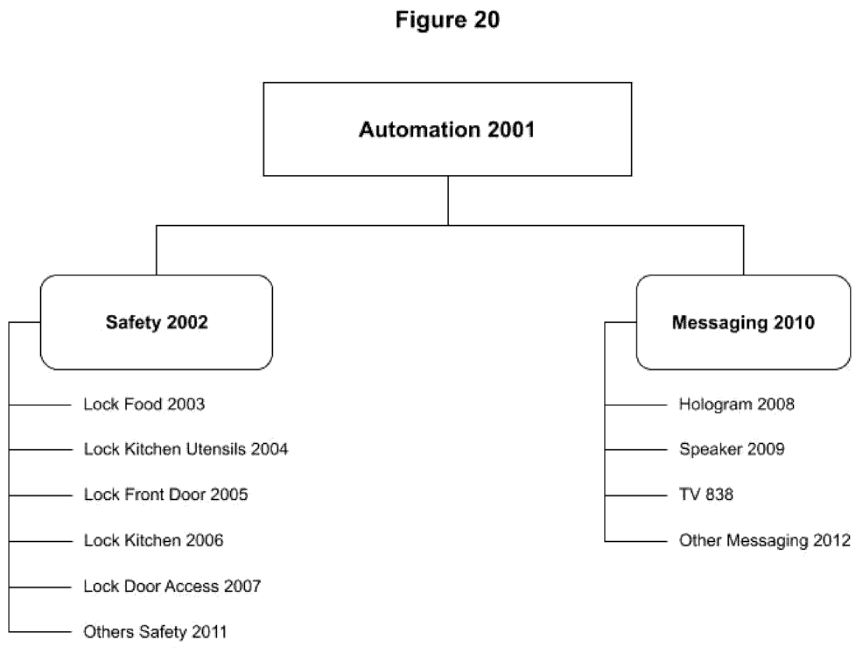

FIG. 20 depicts automation that may be implemented in embodiments of the invention.

FIG. 20 illustrates the potential automation capabilities integrated into embodiments of the invention, emphasizing enhancements in safety and communication systems. The overarching category of Automation 2001 highlights the technological advancements that streamline critical functions, ensuring efficiency, security, and responsiveness.

Safety 2002 is a central focus, addressing various mechanisms to prevent hazards and secure environments. Examples include features such as Lock Food 2003 to restrict access to potentially dangerous or allergenic items, Lock Kitchen Utensils 2004 to limit the use of sharp or hazardous tools, and Lock Front Door 2005 to ensure unauthorized access is prevented. Further enhancements encompass the ability to Lock Kitchen 2006 entirely or restrict Door Access 2007 within specified areas, ensuring safety and privacy. These safety measures are supported by an Other Safety 2011 category, providing a flexible framework for addressing additional security needs specific to various settings.

Messaging 2010 serves as an integral element of automation, designed to ensure effective communication across different mediums. This includes advanced methods like Hologram 2008 for immersive interactions and Speaker 2009 systems to relay auditory notifications or alerts. Other options such as TV 838 enable visual dissemination of critical information, while Other Messaging 2012 ensures the inclusion of innovative or alternative communication technologies to adapt to specific requirements.

By incorporating these automation capabilities, FIG. 20 demonstrates how the invention leverages modern technology to create secure and adaptive environments, enhancing both safety and communication functionalities in diverse applications.

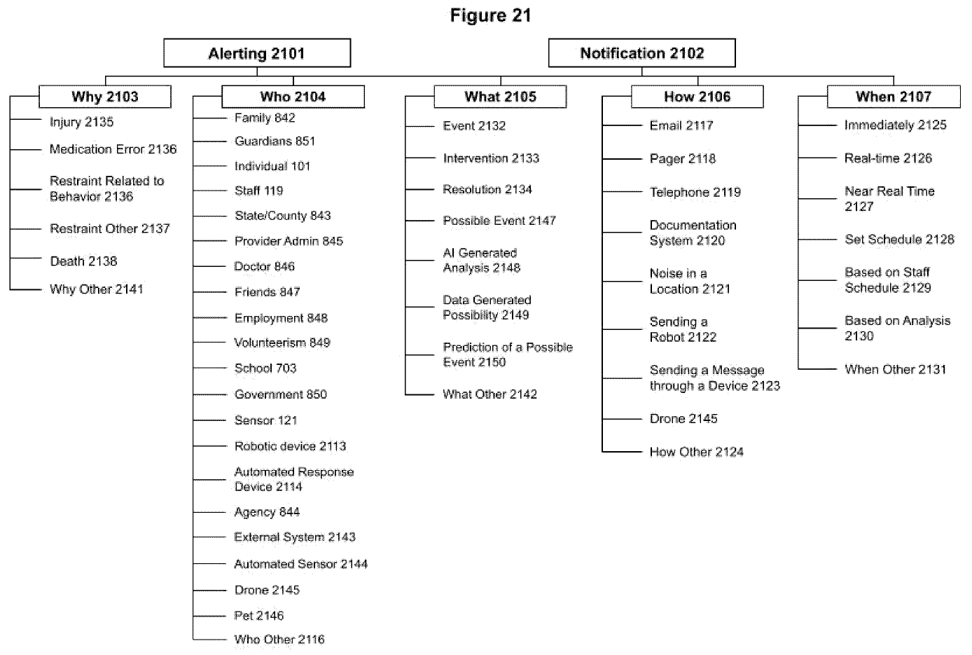

FIG. 21 depicts Alerting 2101 and Notification 2102 that may be implemented in embodiments of the invention.

This figure illustrates on Why 2103 an Alert 2101 and Notification 2102 might be sent, Who 2104 might the recipients of it, What 2105 can the message entail, How 2106 can the recipients be alerted and, When 2107 might be the best possible time to notify regarding an incident that may have or might occur under certain setting.

The contributing factors for Alerting 2101 and sending Notification 2102 might be critical events including but not limited to Injury 2135, Medication Error 2136, Restraint Related to Behavior 2136. For example, individuals might refuse to take Prescribed Drugs 933 independent of any problems with the physical environment they are in.

In circumstances similar to this, the Alert 2101 and Notification 2102 might be sent to a person such as Family 842, Guardians 851, other staff members. Additionally, devices including but not limited to Sensor 121, Robotic device 2113, Automated Response Device 2114, External System 2143, Drone 2145 might also capture details of the incident. The event may be reported to a reporting agency (Agency 844) or a government agency (Government 850).

In case of an incident that might have already occurred, the Alert 2101 and/or Notification 2102 might contain insights of the Event 2132, Intervention 2133, Resolution 2134. On the other hand, if any incident is yet to take place, the recipients might receive alerts such as, but not exclusively on Possible Event 2147, Al Generated Analysis 2148, Data Generated Possibility 2149, Prediction of a Possible Event 2150.

There are many different methods which might be used to send these Alerts 2101 and Notifications 2102, consisting of, but not confined to Email 2117, Pager 2118, Telephone 2119, Documentation System 2120, Noise in a Location 2121, Sending a Robot 2122, Sending a message through a device 2123, Drone 2145.

The timeframe of sending the message to the recipients might vary depending on various predictable and unpredictable reasons. The notification might be sent Immediately 2125, in Real-time 2126, Near real time 2127, Set Schedule 2128, Based on staff schedule 2129, Based on analysis 2130 and so on. For example, an Individual 102 who might be intellectually disabled opens the door in response to continuous pounding on it. As soon as Individual 102 opens the door, some total stranger who's agitated comes into the room which in turn triggers the sensors to send immediate notification, informing about the intruder in the room.

As discussed above with reference to FIGS. 1, 4, 5A-G, and 14-15, and applicable to the scenarios illustrated in FIGS. 14A-F, 15A-F, 16A-D, and 17A-D, events and actions taken may be documented in accordance with the preferred embodiments of this invention. Documentation may take the form of General Event Reports or T-Logs, or other formats.

A General Event Report (GER) documents an incident that can include injury, medication error, restraint, death, or the “other” category which may include things such as a person going to the emergency room, etc. Also preferably included within GERs is the opportunity to use a Witness Report to gather further information on a given event from additional staff members who were present.

Preferably, a GER Resolution module is used to document investigation details, recommendations, involved persons and whether or not the investigation is open or closed for the associated GER. Further, a Witness Report preferably gets created if a person is listed as a Witness on the GER and includes the associated GER, Event and Witness details. In addition, a Multi Individual Event module preferably provides users the ability to link related GER forms into one single form, instead of having to document similar GER forms multiple times.

Many states require incident reports to be submitted in a specific format. Preferably, the option to add State-specific information is provided regarding an incident to a GER form. The State-specific GER forms have been designed according to the policy of different States.

Preferably, users are also able to take printable version of the State specific information regarding an incident.

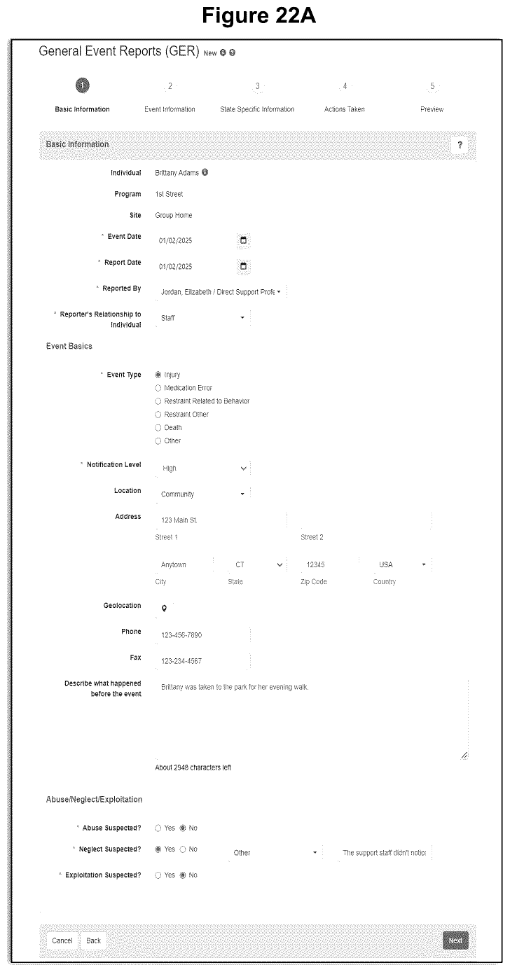

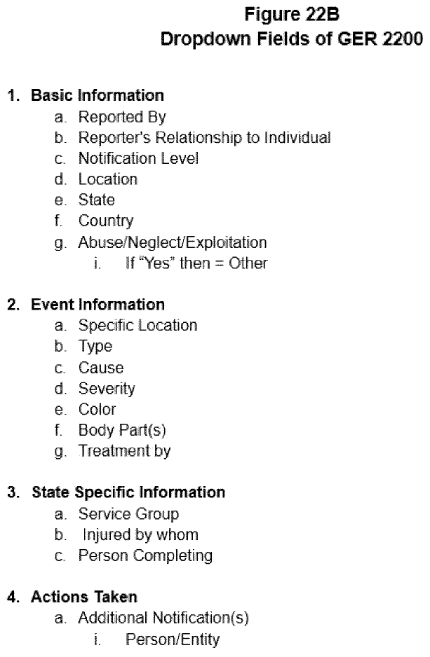

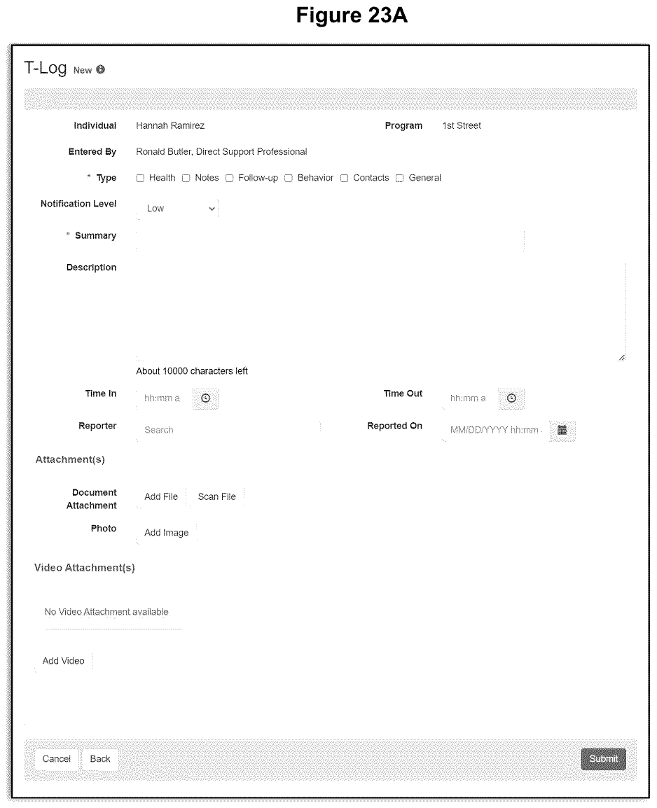

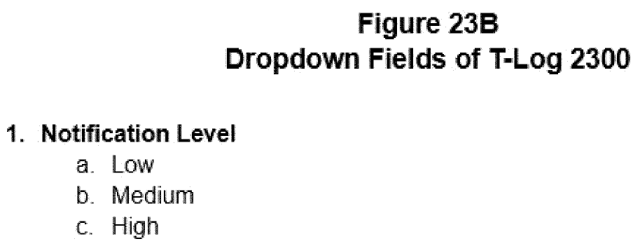

FIG. 22A-23B illustrate preferable GER forms that may be filled in with information generated as part of this invention. In particular, FIG. 22A shows an electronic form format for creating a GER and FIG. 22B shows Dropdown Fields of GER 2200 for receiving information generated as part of this invention.

A T-Log preferably offers a simple and effective way for a user (including agencies and their representatives) to enter and share daily shift notes, case notes, contact notes or logs efficiently. A T-Log preferably allows the collection and communication of day-to-day information and progress notes with other staff members who can add follow-ups and share in a way that complies with HIPAA-type regulations.

FIGS. 23A-23B illustrate preferable T-Log forms that may be filled in with information generated as part of this invention. In particular, FIG. 23A shows a preferable electronic form format for creating a T-Log, and FIG. 23B shows preferable Dropdown Fields of T-Log 2300 for receiving information generated as part of this invention.

Embodiments of this invention may include:
a. Device on a person that recognizes it is not receiving proper information from external sources showing that a monitoring infrastructure if operations and then sends an alert to the person with/wearing the device.
b. Device as above but tied to individuals with caseloads-so you only get this information based on your caseload regarding a specific individual.
c. Device in a room with fundamental non-changeable rules-so instructions from outside the rule would be overruled and force some sort of human or other local interaction.

1. An improvement to the way that computer systems operate to monitor and assist individuals under care by a caregiver, the improvement comprising a HIPAA-compliant method of receiving and recording personal health information data in an electronic format relating to at least one individual in a monitored location from at least two sensors, creating textual descriptions from the data, combining the textual descriptions into a unified textual description and comparing the unified textual description to stored textual descriptions, identifying an event and an action corresponding to the event, and performing the action, the method comprising: performing, by the computer system, the steps of:
a. providing an event database of stored textual descriptions of events;
b. providing an event-to-action database of action plans corresponding to said events;
c. providing a location having at least two sensors for collecting data regarding the individual;
d. collecting data from said sensors;
e. creating respective textual descriptions of said data collected by each of said sensors;
f. combining said textual descriptions into a unified textual description;
g. comparing said unified textual description to at least one said stored textual description;
h. identifying whether said unified textual description matches one of said stored textual descriptions, identifying a matching event, and identifying an action corresponding to said matching event;
i. identifying, if said unified textual description does not match one of said stored textual descriptions, whether said unified text description is similar to a stored textual description, identifying a similar event, and identifying an action corresponding to said similar event;
j. identifying whether said unified text description does not match a stored textual description and whether said unified text description is not similar to a stored textual description, accepting a training input identifying an action corresponding to said non-matched event; and storing said non-matching event and said action corresponding to said non-matching event in said event-to action database; and
k. performing an identified action.

2. The method of claim 1 wherein said identified action is sending an alert to a device.

3. The method of claim 2 wherein said alert is an instruction to a caregiver to provide assistance to the individual.

4. The method of claim 1 wherein said sensors include one or more video cameras and said data includes video information, and further including the step of identifying the individual, said identification step further including performing, by the computer system, the steps of:
a. providing a database storing records of physical attributes of the individual,
b. comparing the stored records of physical attributes of the individual to said video information, and
c. identifying the individual by matching at least one of a face, eye color, and/or hair color in said stored records of physical attributes of the individual with the face, eye color, and/or hair color of the individual under care in said video information.

5. The method of claim 4 wherein said step of identifying a matching event includes identifying the individual.

6. The method of claim 4 wherein said step of identifying a similar event includes identifying the individual.

7. The method of claim 4 wherein said identified action is providing the identification of said event to a caregiver based on said identification of the individual, and further including performing, by the computer system, the steps of:
a. providing a database storing at least one authorization profile associated with the caregiver, wherein the caregiver is associated with one or more roles and one or more caseloads and said caseloads include access privilege information for the individual, wherein said access privilege information included in said caseload includes the identities of individuals to which the caregiver has access,
b. comparing the identity of the individual to the caregiver's authorization profile information, including comparing said identity of the individual to the caregiver's caseload, and
c. providing the identification of said event to the caregiver if said identity of the individual is stored in the caregiver's caseload.

8. The method of claim 1 in which at least one sensor is a smart socket device.

9. The method of claim 8 in which said smart socket device provides data indicating whether one or more network components are active.

10. The method of claim 9 wherein said network components include one or more of electricity, internet, and a communication network.

11. An improved computer system for monitoring and assisting individuals under care by a caregiver, the improvement comprising a HIPAA-compliant system for receiving and recording personal health information data in an electronic format relating to at least one individual in a monitored location from at least two sensors, creating textual descriptions from the data, combining the textual descriptions into a unified textual description and comparing the unified textual description to stored textual descriptions, identifying an event and an action corresponding to the event, and performing the action, the system comprising:
a. a location having at least two sensors for collecting data regarding the individual;
b. a computer system having a memory and a processor, and including:
i. an event database of stored textual descriptions of events, and
ii. an event-to-action database of action plans corresponding to said events;
c. a computer program stored in said memory and adapted to run on said processor, configured to:
i. collect data from said sensors;
ii. create respective textual descriptions of said data collected by each of said sensors;
iii. combine said textual descriptions into a unified textual description;
iv. compare said unified textual description to at least one said stored textual descriptions;
v. identify whether said unified textual description matches one of said stored textual descriptions, identify a matching event, and identify an action corresponding to said matching event;
vi. identify, if said unified textual description does not match one of said stored textual descriptions, whether said unified text description is similar to a stored textual description, identify a similar event, and identify an action corresponding to said similar event;
vii. identify whether said unified text description does not match a stored textual description and whether said unified text description is not similar to a stored textual description, accept a training input identifying an action corresponding to said non-matched event; and store said non-matching event and said action corresponding to said non-matching event in the event-to action database; and
viii. perform said identified action.

12. The system of claim 11 further including a device, wherein said identified action is sending an alert from said computer system to said device.

13. The system of claim 12 wherein said alert is an instruction to a caregiver to provide assistance to the individual.

14. The system of claim 11 wherein the sensors include one or more video cameras and said data includes video information, wherein said computer system further includes a database storing records of physical attributes of the individual, and wherein said computer program is further configured to identify the individual by:
a. comparing said stored records of physical attributes of the individual to said video information, and
b. identifying the individual by matching at least one of a face, eye color, and/or hair color in said stored records of physical attributes of the individual with face, eye color, and/or hair color of the individual in the video information.

15. The system of claim 14 wherein said computer program is configured to identify said matching event by identifying the individual.

16. The system of claim 14 wherein said computer program is configured to identify said similar event by identifying the individual.

17. The system of claim 14 wherein
said action is providing said identification of an event to a caregiver based on said identification of the individual,
said computer system further includes a database storing at least one authorization profile associated with the caregiver, wherein the caregiver is associated with one or more roles and one or more caseloads and said caseloads include access privilege information for the individual, wherein said access privilege information included in said caseload includes the identities of individuals to which the caregiver has access, and
said computer program is configured to:
a. compare the identity of the individual to the caregiver's authorization profile information, including comparing the identity of the individual to the caregiver's caseload; and
b. provide said identification of said event to the caregiver if said identity of the individual is stored in the caregiver's caseload.

18. The system of claim 11 in which at least one sensor is a smart socket device.

19. The system of claim 18 in which said smart socket device is configured to provide data indicating whether one or more network components are active.

20. The system of claim 19 wherein said network components include one or more of electricity, internet, and a communication network.
